(19) United States
(12) Patent Application Publication
(10) Pub. No.: US 2024/0102048 A1
(43) Pub. Date: Mar. 28, 2024

(54) GENE THERAPY FOR RETINAL DISEASES

(71) Applicant: Greffex, Inc., Houston, TX (US)

(72) Inventor: Uwe D. STAERZ, Denver, CO (US)

(21) Appl. No.: 18/264,104

(22) PCT Filed: Feb. 14, 2022
(86) PCT No.: PCT/US2022/016318

§ 371 Date: Aug. 3, 2023

Related U.S. Application Data
(60) Provisional application No. 63/149,122, filed on Feb. 12, 2021.

(51) Int. Cl. C12N 15/86; C07K 14/005

(52) U.S. Cl. CPC C12N 15/86; C07K 14/005; C12N 2710/10322; C12N 2710/10343; C12N 2830/003; C12N 2830/008

(57) ABSTRACT

A viral vector comprises a transgene construct. In some embodiments, the transgene construct is a DNA sequence comprising a promoter element, a retinal gene's open reading frame being longer than 10 kb, and a terminator sequence. In other embodiments, the transgene construct is a DNA segment of retinal genes longer than 10 kb.

### CROSS REFERENCE TO RELATED APPLICATIONS

This application claims priority to and is a non-provisional application of Provisional Application No. 63/149,122, filed on Feb. 12, 2021, which is incorporated herein by reference in its entirety.

### SUBMISSION OF SEQUENCE LISTING

A sequence listing containing the file named Sequence Listing which is 110 kilobytes in size (measured in MS-Windows) and was created on Feb. 11, 2022, is provided herewith and is incorporated by reference in its entirety.

### FIELD

The disclosure relates to gene therapy to treat Usher syndrome type II A, Usher syndrome type 2C, retinitis pigmentosa 39, retinis pigmentosa 25, Wagner syndrome, or age related macular degeneration.

### BACKGROUND

Usher syndrome type 2 is a leading cause of combined blindness and deafness. Retinitis pigmentosa (RP) is a leading cause of inherited blindness. Usher syndrome 2A (Ush2A) mutations are estimated to cause around 20% of retinitis pigmentosa cases, and may be the single biggest cause. Syndromic Usher 2A syndrome involves loss of hearing at an early age, with vison loss occurring in early to mid-adulthood. Non-syndromic blindness can be caused by Ush2A mutations in which hearing is normal but vision decline still occurs.

Usher syndrome type 2C (Ush2C), RP 39, RP 25, Wagner syndrome, and hemicentin mutations causing age related macular degeneration likewise involve photoreceptor cell loss, but are much rarer diseases than Usher type 2A. All of these diseases, however, lead to loss of vision and involve mutations in genes too large for conventional gene therapy using adeno-associated virus.

Viral gene therapy vectors found their first major clinical success in the treatment of an X-linked form of severe combined immune deficiency. The retroviral vector achieved long-term immune reconstitutions, yet caused life-threatening acute lymphatic leukemias in 30% of subjects. Other vector systems, such as the adeno-associated virus (AAV) ones, have advanced in recent years despite having a limited capacity and being linked to the development of hepatocellular carcinomas. The chromosomal integration of lentivirus vectors allows persistent transgene expression, but may cause insertional mutagenesis. Adenovirus vectors (Ad vectors) transduce both dividing and non-dividing cells, leading to quick and efficient transgene expression. In their first iterations, Ad vectors were hampered by induced and pre-existing immune responses. To minimize immune and inflammatory responses, all endogenous Ad genes needed to be gutted from the Ad vector to create fully deleted (fd) Ad vectors. Capsid modifications and anti-inflammatories also reduced Ad-associated inflammation, and gene therapy approaches became possible.

Gene therapy approaches to Usher type 2 syndrome caused by Ush2A mutations have been proposed involving altering RNA splicing to skip the affected exon (ProQR's QR-421a) or using CRISPR/Cas9 editing to fix the mutation. These approaches would require a treatment tailored to each individual mutation or mutation region. As of 2018, UMD-USHbase has listed 3083 mutations in Ush2A associated with blindness. The most common mutations account for only 38% of pathogenic mutations. Supplying a normal copy of the entire gene would be an off-the-shelf solution that could potentially solve all Ush2A mutations. Additionally, potential side effects of CRISPR editing in vivo include cancer and increased photoreceptor death. These side effects would be avoided with an episomal copy of the gene. Similarly, Ush2C, RP39, RP25, Wagner syndrome and hemicentin mutations causing age related macular degeneration could be solved with one gene therapy approach for each gene, rather than having to tailor thousands of individual solutions.

Additionally, it may be advantageous for retinal gene therapy to take a genomic editing approach due to dominant negative effects of mutations in genomic copies of the genes, deletions of large non-coding regions of genes important for retinal function, or to add a compensating gene construct to fix other problems. In such cases, it may be important to fix the problem using homology-independent targeted integration (HITI) or homologous recombination (HR) using a donor/template sequence longer than the 5 kb adeno-associated virus (AAV) is capable of Adenoviruses can supply a template up to 30 kb in length and can additionally fit the machinery required for the CRISPR editing in the same vector, increasing the efficiency of the treatment.

Moreover, the gene length of the important transcript in the case of Ush2A is around 15,000 (15 kb) base pairs. The maximum capacity of AAV is around 4,000 base pairs. Lentivirus is another common gene therapy vector (despite safety concerns of genomic integration) but has a maximum of 10,000 base pairs.

### SUMMARY

In an embodiment the present disclosure provides a viral vector. In accordance with embodiments of the present disclosure, the viral vector comprises a transgene construct, wherein the transgene construct is one of (a) a DNA sequence comprising a promoter element, a retinal gene's open reading frame being longer than 10 kb, and a terminator sequence, and (b) a DNA segment of retinal genes longer than 10 kb.

In an embodiment, the vector is fully deleted of endogenous genes aside from ITR sequences. In another embodiment, the vector is deleted of enough endogenous adenoviral genes to make up to 10 kb of space for the cargo sequence. In a further embodiment, the viral vector is a fully or partially gutted mimivirus.

In another embodiment, the viral vector is a fully or partially gutted adenovirus. In an embodiment, the viral vector is encapsidated in a capsid based on adenovirus type 5. In a further embodiment, the viral vector is encapsidated in a capsid based on adenovirus type 6. In still a further embodiment, the viral vector is encapsidated in a capsid based on adenovirus and modified to remove RGD sequences from the capsid proteins. In yet another embodiment, the viral vector is encapsidated in a capsid based on adenovirus in families A-G. In a still further embodiment, the transgene construct is (a) a DNA sequence comprising a promoter element, a retinal gene's open reading frame being longer than 10 kb, and a terminator sequence.

In an embodiment, the promoter element is a human rhodopsin kinase promoter. In another embodiment, the promoter element is a human rhodopsin kinase promoter. In accordance with a further embodiment, the promoter element is a mouse rhodopsin kinase promoter. In a further embodiment, the promoter element is a ubiquitous CMV promoter. In still another embodiment, the promoter element is an upstream non-coding sequence of Usherin gene containing the native promoter of the Usherin gene. In another embodiment, the promoter element is a synthetic a transducin alpha-subunit promoter. In still a further embodiment, the promoter element is an inter-photoreceptor retinoid-binding protein promoter and a minimal sequence of the human transducin alpha-subunit promoter. In yet a further embodiment, the promoter element is a chemically controlled tet inducible promoter. In another embodiment, the promoter element is a chemically inhibited promoter. In another embodiment, the promoter element is a human L-opsin promoter. In a further embodiment, the promoter element is a human retinoschisin proximal promoter and the human interphotoreceptor retinoid-binding protein enhancer. In still a further embodiment, the promoter element is specific to photoreceptors.

In an embodiment, the transgene construct is a nucleotide sequence which translates to Ush2A. In another embodiment, the transgene construct is a nucleotide sequence which translates to ADGV1. In a further embodiment, the transgene construct is a nucleotide sequence which translates to eyes shut homolog. In yet another embodiment, the transgene construct is a nucleotide sequence which translates to Hemicentin. In still a further embodiment, the transgene construct is a nucleotide sequence which translates to Versican.

In an embodiment, the transgene construct is (b) a DNA segment of retinal genes longer than 10 kb. In an embodiment, the DNA segment is at least a segment of the Ush2A gene. In another embodiment, the DNA segment is at least a segment of the ADGV1 gene. In still another embodiment, the DNA segment is at least a segment of the eyes shut homolog gene. In yet a further embodiment, the DNA segment is at least a segment of the Hemicentin gene. In still another embodiment, the DNA segment is at least a segment of the Versican gene.

In an embodiment, the transgene construct is (b) a DNA segment of retinal genes longer than 10 kb.

In an embodiment, the disclosure provides a composition. In accordance with embodiments of the present disclosure, the composition comprises a viral vector and a packing plasmid, the viral vector containing a transgene construct selected from (a) a DNA sequence comprising a promoter element, a retinal gene's open reading frame being longer than 10 kb, and a terminator sequence, and (b) a DNA segment of retinal genes longer than 10 kb.

In an embodiment, the present disclosure provides a method of treating retinal disease. In accordance with embodiments of the present disclosure, the method of treating retinal disease comprises providing a viral vector comprising a transgene construct, wherein the transgene construct is one of (a) a DNA sequence comprising a promoter element, a retinal gene's open reading frame being longer than 10 kb, and a terminator sequence, and (b) a DNA segment of retinal genes longer than 10 kb.

In an embodiment, the retinal disease is selected from the group consisting of Usher syndrome type II A, Usher syndrome type 2C, retinitis pigmentosa 39, retinis pigmentosa 25, Wagner syndrome, and age related macular degeneration.

### BRIEF DESCRIPTION OF THE SEQUENCE LISTING

SEQ ID NO. 1: shows the DNA sequence for USH2A.

SEQ ID NO. 2: shows the DNA sequence for USH2A behind a CMV promoter.

SEQ ID NO. 3: shows the DNA sequence for USH2A behind a rhodopsin kinase promoter.

### BRIEF DESCRIPTION OF THE DRAWINGS

FIG. 1 illustrates the components of an adenovirus vector. In particular, FIG. 1A illustrates the design of a vector genome model in accordance with embodiments of the present disclosure, FIG. 1B illustrates the design of a pPaC packaging expression plasmid in accordance with embodiments of the present disclosure, FIG. 1C illustrates vector encapsidation by co-transfection into host cells in accordance with embodiments of the present disclosure, and FIG. 1D illustrates the release of a packaged vector in accordance with embodiments of the present disclosure.

FIG. 2 illustrates the transduction of test cells with a hemophilia 8 gene transfer vector in accordance with embodiments of the present disclosure.

FIG. 3 illustrates transfection of mouse retina cells to test for specific gene transcription.

FIG. 4 illustrates the construction of a USH2A vector by (1) forced cloning and (2) stuffing by recombineering.

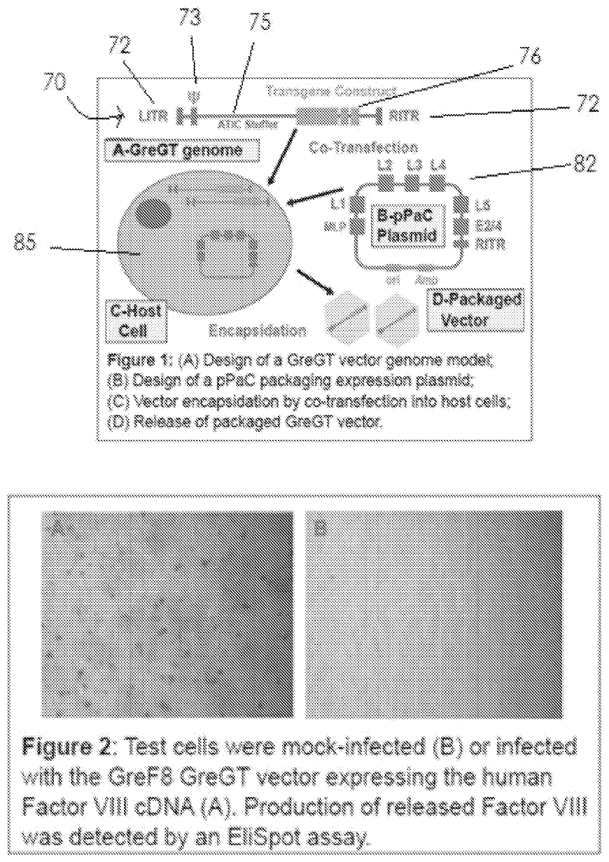

### DETAILED DESCRIPTION

Before any embodiments of the present disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is understood that the phraseology and terminology used herein is for the purpose of description and should not be regarded as limiting. The use of “including,” “comprising” or “having” and variations thereof herein is meant to encompass the items listed thereafter and equivalents thereof as well as additional items. The use of “including essentially” and “consisting essentially of” and variations thereof herein is meant to encompass the items listed thereafter, as well as equivalents and additional items provided such equivalents and additional items to not essentially change the properties, use or manufacture of the whole. The use of “consisting of” and variations thereof herein is meant to include the items listed thereafter and only those items.

With reference to the drawings, like numbers refer to like elements throughout. It will be understood that, although the terms first, second, etc., may be used herein to describe various elements, components, regions, and/or sections, these elements, components, regions and/or sections should not be limited by these terms. These terms are used only to distinguish one element, component, region and/or section from another element, component, region and/or section. Thus, a first element, component, region or section could be termed a second element, component, region or section without departing from the disclosure.

The numerical ranges in this disclosure are approximate, and thus may include values outside of the range unless otherwise indicated. Numerical ranges include all values from and including the lower and the upper values (unless specifically stated otherwise), in increments of one unit, provided that there is a separation of at least two units between any lower value and any higher value. As an example, if a compositional, physical or other property, such as, for example, amount of a component by weight, etc., is from 10 to 100, it is intended that all individual values, such as 10, 11, 12, etc., and sub ranges, such as 10 to 44, 55 to 70, 97 to 100, etc., are expressly enumerated. For ranges containing explicit values (e.g., a range from 1, or 2, or 3 to 5, or 6, or 7), any subrange between any two explicit values is included (e.g., the range 1-7 above includes subranges 1 to 2; 2 to 6; 5 to 7; 3 to 7; 5 to 6, etc.). For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure.

Spatial terms, such as “beneath,” “below,” “lower,” “above,” “upper,” and the like, may be used herein for ease of description to describe one element's or feature's relationship to another element(s) or feature(s) as illustrated in the figures. It will be understood that the spatially relative terms are intended to encompass different orientations depending on the orientation in use or illustration. For example, if a device in the figures is turned over, elements described as “below” or “beneath” other elements or features would then be oriented “above” the other elements or features. Thus, the exemplary term “below” can encompass both an orientation of above and below. A device may be otherwise oriented (rotated 90° or at other orientations) and the spatially relative descriptors used herein interpreted accordingly.

As used herein, the term “and/or” includes any and all combinations of one or more of the associated listed items. For example, when used in a phrase such as “A and/or B,” the phrase “and/or” is intended to include both A and B; A or B; A (alone); and B (alone). Likewise, the term “and/or” as used in a phrase such as “A, B and/or C” is intended to encompass each of the following embodiments” A, B and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure belongs. Generally, the nomenclature used herein and the laboratory procedures in cell culture, molecular genetics, and nucleic acid chemistry and hybridization described below are those well-known and commonly employed in the art. Standard techniques are used for recombinant nucleic acid methods, polynucleotide synthesis, and microbial culture and transformation (e.g., electroporation, lipofection). Generally, enzymatic reactions and purification steps are performed according to the manufacturer's specifications. The techniques and procedures are generally performed according to conventional methods in the art and various general references (see generally, Sambrook et al. Molecular Cloning: a Laboratory Manual, 2d ed. (1989) Cold Spring Harbor Laboratory Press, Cold Spring Harbor, N.Y., which is incorporated herein by reference), which are provided throughout this document. Units, prefixes, and symbols may be denoted in their SI accepted form. Unless otherwise indicated, nucleic acids are written left to right in 5′ to 3′ orientation; amino acid sequences are written left to right in amino to carboxyl orientation, respectively. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5.sup.th edition, 1993).

The term “adenovirus vector” as used herein includes any genetic construct or viral constructs that are based on an adenovirus and used to transfer genetic material. The terms “deleted adenovirus” or “deleted adenovirus vectors” as used herein include any and all adenoviruses or adenovirus vectors which have one or more endogenous genes or gene fragments deleted from it. In contrast, the terms “fully deleted adenovirus” and “fully deleted adenovirus vector” as used herein include any and all adenoviruses and adenovirus vectors from which all endogenous adenoviral genes and genetic material are deleted with the exception of the internal terminal repeats (ITRs) and the packaging signal (ψ). The term “adenoviral vector genome” as used herein includes the genetic material that is found in the adenovirus vector.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A transcription termination sequence may be located 3′ to the coding sequence. Transcription and translation of coding sequences are typically regulated by “control elements,” including, but not limited to, transcription promoters, transcription enhancer elements, Shine and Delagamo sequences, transcription termination signals, polyadenylation sequences (located 3′ to the translation stop codon), sequences for optimization of initiation of translation (located 5′ to the coding sequence), and translation termination sequences.

The term “delete” or “deleted” as used herein means expunging, erasing, or removing.

The terms “deleted Ad (virus) vector” and “gutted-,” “mini-,” “deleted-,” “.DELTA.,” or “pseudo-vectors,” as used herein, refer to a linear vector module with ITRs. These vectors can also code for some structural and/or nonstructural gene sequences and/or one or more genes of interest or transgenes.

The term “expression” refers to the transcription and/or translation of an endogenous gene, transgene or coding region in a cell.

A “ gene delivery vector,” “GDV,” “gene transfer vector,” or “gene transfer vehicle” is a composition including a packaged vector module of the present disclosure.

As used herein, the term “gene expression construct” refers to a promoter, at least a fragment of a gene of interest, and a polyadenylation signal sequence. A vector module of the present disclosure may comprise a gene expression construct.

The terms “gene of interest,” “GOT,” and “transgene,” as used herein, refer to genes that code for genes whose function is of medical interest and may not be a natural flavivirus gene. A gene of interest can be one that exerts its effect at the level of RNA or protein. Examples of genes of interest include, but are not limited to, therapeutic genes, immunomodulatory genes, virus genes, bacterial genes, protein production genes, inhibitory RNAs or proteins, and regulatory proteins.

A “gene sequence” refers to the order of nucleotides. A gene sequence can be regulatable. Regulation of gene expression can be accomplished by one of (1) alteration fo gene structure: site-specific recombinases (e.g., Cre based on the Cre-loxP system) can activate gene expression by removing inserted sequences between the promoter and the gene; (2) changes in transcription: either by induction (covered) or by relief of inhibition; (3) changes in mRNA stability, by specific sequences incorporating in the mRNA or by siRNA; and (4) changes in translation, by sequences in the mRNA. Deleted adenoviruses are also called “high-capacity” adenoviruses. These deleted adenoviruses can accommodate up to 33 kb of genetic sequences.

The term “heterologous” is used for any combination of DNA sequences that is not normally found intimately associated with nature.

The term “homology” refers to the existence of shared ancestry between a pair of structure or genes.

A “host cell” or “packaging cell” is a cell that is able to package adenovirus or adenovirus vector genomes or modified genomes to produce viral particles. It can be engineered to provide a missing gene product or its equivalent. Thus, packaging cells are able to package the adenovirus genomes into the adenovirus particle. The production of such particles requires that the genome be replicated and that those proteins necessary for assembling an infectious virus are produced. The particles also can require certain proteins necessary for the maturation of the viral particle. Such proteins can be provided by a vector, a packaging construct or by the packaging cell. Exemplary host cells (HCs) that may be used to make ap packaging cell line according to the present disclosure include, but are not limited to, A549, HeLa, MRCS, W138, CHO cells, Vero cells, human embryonic retinal cells, or any eukaryotic cells, as long as the host cells are permissive for growth of adenoviruses. Some host cell lines include adipocytes, chondrocytes, epithelial, fibroblasts, glioblastoma, hepatocytes, keratinocytes, leukemia, lympohoblastoid, monocytes, macrophages, myoblasts, and neurons. Other cell types include, but are not limited to, cells derived from primary cell cultures, e.g., human primary prostate cells, human embryonic retinal cells, human stem cells. Eukaryotic diploid and aneuploid cell lines are included within the scope of the disclosure. The packaging cell must be one that is capable of expressing the products of the different constructs described in here at the appropriate level for those products in order to generate a high titer stock of recombinant virus vectors.

An “immune response” is an acquired immune response, such as a cellular or humoral immune response.

The terms “introducing” or “transfection,” as used herein, refer to delivery of an expression vector to a host cell. A vector may be introduced into the cell by transfection, which typically means insertion of heterologous DNA or RNA into a cell by physical means (e.g., calcium phosphate transfection, electroporation, microinjection or lipofection); infection, which typically refers to introduction by way of an infectious agent, i.e., a virus; or transduction, which typically means stable infection of a cell with a virus or the transfer of genetic material from one microorganism to another by way of a viral agent (e.g., a bacteriophage). A vector may be a plasmid, virus or other vehicle.

The term “packaging construct” or “packaging expression plasmid” refers to an engineered plasmid construct of circular, double-stranded DNA molecules, wherein the DNA molecules include at least a subset of adenovirus structural or nonstructural genes under control of a promoter. The “packaging construct” does not comprise more than one ITR or genetic information to enable independent virus replication to produce infections, viral particles and/or efficient packaging of this genetic material being packaged into a viral particle.

The term “plasmid,” as used herein, refers to an extra-chromosomal DNA molecule separate from the chromosomal DNA, which is capable of replication independently of the chromosomal DNA. In many cases, it is circular and double-stranded.

The term “promoter” means a regulatory region of DNA that facilitates the transcription of a particular gene. Promoters usually comprises a TATA box capable of directing RNA polymerase II to initiate RNA synthesis at the appropriate transcription initiation site for a particular coding sequence. A promotor may additionally comprise other recognition sequences generally positioned upstream or 5′ to the TATA box, referred to as upstream promoter elements, which influence the transcription initiation rate.

The term “transfection” refers to the instruction into a cell genetic material as DNA or RNA (for example, introduction of an isolated nucleic acid molecule or a construct of the present disclosure). The term “transduction” as used herein refers to the introduction into a cell DNA either as DNA or by means of a GDV of the present disclosure. A GDV of the present disclosure can be transduced into a target cell.

The term “vector” refers to a nucleic acid used in infection of a host cell and into which can be inserted a polynucleotide. Vectors are frequently replicons. Expression vectors permit transcription of a nucleic acid inserted therein. Some common vectors include, but are not limited to, plasmids, cosmids, viruses, phages, recombinant expression cassettes, and transposons. The term “vector” may also refer to an element which aids in the transfer of a gene from one location to another.

The term “vector module” refers to an adenovirus genetic composition that is packaged in an adenovirus virion.

In accordance with embodiments of the present disclosure, an adenovirus vector is provided. The adenovirus vector comprises a transgene construct which is a DNA segment or DNA sequence. In an embodiment, the adenovirus vector is contained in a capsid. In an embodiment, the capsid is provided as gene therapy in the treatment of Usher syndrome type II A, Usher syndrome type 2C, retinitis pigmentosa 39, retinitis pigmentosa 25, Wagner syndrome, or age related macular degeneration, or in the prevention of retinal photoreceptor degeneration caused by loss of Ush2A, ADGRV1, eyes shut homolog, hemicentin, or versican(VCAN) protein due to mutations.

### Viral Vectors

The vectors of the present disclosure are based on a virus selected from the group consisting of adenovirus and mimivirus. Preferably, the viral vector is based on adenovirus. The viral vectors used in the present disclosure are designed as fully deleted (fd) viruses, and more particularly, fd adenoviruses, that are packaged in the absence of helper viruses. The viral vector is one of two independently modifiable components of the capsid, the other being the circular packaging expression plasmid.

In an embodiment, the viral vector is an Ad vector.

In an embodiment, the viral vector is an Ad vector, fully deleted, and free of helper viruses.

In an embodiment, the adenovirus vector 70, preferably fd adenovirus vector, is capable of receiving gene constructs and carry inverted terminal repeat sequences (ITRs) 72, 72 and a packaging signal (Ψ) 73, as shown in FIG. 1. The deleted endogenous genes are replaced with size-compensating stuffers 75. In the embodiment shown, these stuffers 75 are prepared from fragments of the human gene 5-aminoimidazole-4-carboxamide ribonucleotide formyltransferase gene (ATIC). In further embodiments, other stuffer sequences, such as, but not limited to, human hypoxanthine-guanine phosphoribosyltransferase, can be used.

A fd base vector genome module is engineered for transgene constructs 76 of different lengths. In some embodiments, the fd base vector genome module is engineered for transgene constructs from 1 kb, or 5 kb, or 10 kb to 20 kb, or 25 kb, or 30 kb, or 35 kb, and preferably from 10 kb, or greater than 10 kb, or 12 kb, or 15 kb, or 18 kb, or 20 kb to 22 kb, or 25 kb, or 28 kb, or 30 kb, or 32 kb, or less than 35 kb.

In an embodiment, the transgene construct 76 is a DNA sequence comprising a promotor element, a retinal gene's open reading frame longer than 10 kb, and a terminator sequence.

In an embodiment, the promoter is specific to photoreceptors. Non-limiting examples of photoreceptor specific promoters include, but are not limited to, a human rhodopsin kinase promoter, a mouse rhodopsin kinase promoter, a ubiquitous CMV promoter, a ubiquitous CAG promoter, an upstream non-coding sequence of Usherin gene containing the native promoter of the Usherin gene, a synthetic a transducing alpha-subunit promoter, an inter-photoreceptor retinoid-binding protein promotor, a minimal sequence of the human transducin alpha-subunit promoter, a chemically controlled tet inducible promoter, a human L-opsin promoter, a retinoschisin proximal promoter, a human interphotorceptor retinoid-bindin protein enhancer, and combinations thereof.

In a particular embodiment, the promoter is selected from the group consisting of human rhodopsin kinase promoter, mouse rhodopsin kinase promoter, ubiquitous CMV promoter, ubiquitous CAG promoter, upstream non-coding sequence of Usherin gene containing the native promoter of the Usherin gene, synthetic a transducin alpha-subunit promoter, inter-photoreceptor retinoid-binding protein promoter and a minimal sequence of human transducin alpha-subunit promoter, a chemically inhibited promoter, human L-opsin promoter, and human retinoschisin proximal promoter and human interphotoreceptor retinoid-binding protein enhancer.

In an embodiment, the retinal gene's open reading frame is a nucleotide sequence. In an embodiment, the nucleotide sequence translates to a photoreceptor specific protein. Non-limiting examples of nucleotide sequences include nucleotide sequences that translate to a protein encoded by a gene selected from Ush2A, ADGV1, eyes shut homolog, Hemicentin, Versican, and combinations thereof. In an embodiment, the nucleotide sequence is translated to a protein encoded by a gene selected from the group consisting of Ush2A, ADGV1, eyes shut homolog, Hemicentin, and Versican.

In an embodiment, the transgene construct is a DNA segment of retinal genes longer than 10 kb. Non-limiting examples of suitable DNA segments of retinal genes longer than 10 kb include genomic sequences in the Ush2A gene, genomic sequences in the ADGV1 gene, genomic sequences in the eyes shut homolog gene, genomic sequences in the Hemicentin gene, genomic sequences in the Versican gene, and combinations thereof.

Different transgene expression cassettes have been successfully moved into the fd base genome vector module. Due to the episomal location of the Ad vector genome, recombinations into the cell genome are not expected and have not been observed.

### Packing Plasmid

In an embodiment, the viral vector is provided with a packing plasmid.

The circular packaging expression plasmids (pPac) are developed to in trans provide the late genes (L1, L2, L3, L4, L5) together with the early genes E2 and E4, as shown in FIG. 1. The LITR, the packaging signal (ψ), the early genes E1, E3 and protein IX genes are absent from the circular pPac.

In an embodiment, Different packaging plasmids encapsidate vector modules into capsids of different serotypes, such as Ad2 (pPaC2), Ad5 (pPaC5), Ad6 (pPaC6), Ad4 (pPaE4) and Ad35 (pPaB35).

### Transfection

In an embodiment, the viral vector and packing plasmid are transfected into a packaging cell.

In an embodiment, a packaging cell may contain one or more viral vectors and one or more plasmids. In a preferred embodiment, a packaging cell comprises at least one, preferably two or more, and more preferably three or more viral vectors and one packing plasmid.

Referring still to FIG. 1, the viral vectors 70 and plasmid 82 are introduced via co-transfection into a eukaryotic host cell, or packaging cell 85. In the particular embodiment shown, the viral vectors 70 and plasmid 82 are co-transfected into the packaging cell 85 using an optimized standardized one-week co-transfection protocol.

In an embodiment, the viral vector is an adenovirus vector, particularly a fd adenovirus vector, and the packaging cell is derived from cell lines such as, but not limited to, human embryonic kidney cells (HEK293) and PerC.6 cells. The packaging cell necessary to package fd adenovirus vectors must be modified to express the genes coded within the E1 region of an adenoviral vector. In a particular embodiment, the packaging cell is an HEK293-derived Q7 packaging cell modified to express the genes coded within the El region of an adenoviral vector.

### Capsid

In accordance with embodiments of the present disclosure, the packing cell 85 containing the viral vector 70 and plasmid 82 are encapsidated, as shown in FIG. 1.

The packaging cell 85, containing the viral vectors 70 and plasmid 82, is delivered in capsids of serotypes of the adenovirus or mimivirus. In an embodiment, the capsid is based on adenovirus and modified to remove RGD sequences from the capsid proteins. In an embodiment, the capsid is based on adenovirus families A-G. In an embodiment, the viral vector is delivered in capsids of the Ad2, Ad5, Ad6 and Ad35 serotypes of the adenovirus, and combinations thereof. In an embodiment, the viral vector is delivered in capsids of the Ad6 serotype.

In the embodiment provided, the fd vector genome modules are encapsidated by standardized co-transfection protocols of a vector genome module and a packaging expression plasmid. Current technology provides for 2,000 packages viral genomes per cell, and technological advancement may increase the production efficiency in the future. The packages vectors are then purified by column chromatography.

### Treatment of Retinal Diseases

In an embodiment, the viral vector is provided for the treatment of retinal diseases caused by a genetic mutation. A viral vector may be in accordance with any embodiment, or combination of embodiments, described herein. In a further embodiment, the viral vector is provided as a composition, the composition comprising at least one viral vector and at least one packing plasmid. In another embodiment, a packaging cell is provided, wherein the packaging cell contains the at least one viral vector and at least on packing plasmid. In a still further embodiment, a capsid is provided, wherein the capsid contains the at least one viral vector and, preferably, the at least one packing plasmid. The viral vector, packing plasmid, and packaging cell may be in accordance with any embodiment, or combination of embodiments, described herein. In a further embodiment, the packaging cell containing the viral vector and packing plasmid is encapsidated in a capsid.

In an embodiment, the viral vector, composition, packaging cell and/or capsid is injected into a subject's eyes, preferably a mammal subject, and more preferably a human subject. The viral vector is injected into the eyes either subretinally or intravitreally to infect photoreceptors, causing them to express the protein for which the transgene construct encodes. In a further embodiment, the viral vector is provided with a packing plasmid. The viral vector and packing plasma may be transfected into a packaging cell. The packaging cell may be encapsidated into a capsid. The viral vector, packing plasmid, packaging cell and capsid may be, independent, according to any embodiment or combination of embodiments provided herein.

### Examples and Case Studies

The methods and compositions discussed herein allow correction of mutations causing retinal disease, preventing photoreceptor death and blindness. Methods and compositions provided herein prevent the progression of, for example, Usher syndrome type II A, Usher syndrome type 2C, retinitis pigmentosa 39, retinitis pigmentosa 25, Wagner syndrome, or age related macular degeneration due to hemicentin mutations.

The viral vectors for gene therapies described herein is specifically engineered when large transgenes need to be delivered. For example, a viral vector may deliver the full-length human coagulation factor VIII cDNA (7 kb) driven from a CMV immediate early promoter/enhancer together with an immune suppressive molecule for a total expression cassette of about 12 kb. Fibroblasts transduced with such a vector produced the factor VIII as a focus and a shadow.

In an embodiment, the broadly active CMV immediate early promoter/enhancer was used as a standard to gauge the activity of the photoreceptor specific human rhodopsin kinase (RK) promoter, for which a highly potent variant has been identified and which drives gene expression both in cones and rods. Because the universal CMV immediate early promotor/enhancer promoter functions in microglia cells and therefore may induce detrimental inflammatory response (FIG. 3), it will only be used as a gauge of activity of RK. The enhanced green fluorescent protein (GFP) is the used to test the promoter activities of different vectors.

The enhanced green fluorescent protein (GFP) gene was used as a test gene in in vivo electroporation studies. Approximately 0.5 μl of a solution of the respective purified plasmid DNA in PBS (5 mg/ml) was injected into the subretinal space of newborn mice using a Hamilton syringe with a 32-gauge blunt-ended needle. The electroporation was conducted by applying five square pulses using a pulse generator. At day 21 post electroporation, the mice were sacrificed and the retinal sections analyzed for vector uptake. As exemplified in FIG. 3, both promoters drove GFP expression in photoreceptor cells to comparable levels. The broadly reactive CMV promoter also induced GFP transgene expression in the microglia. Vectors using the broadly active CMV promoter may therefore be more prone to initiate inflammatory responses.

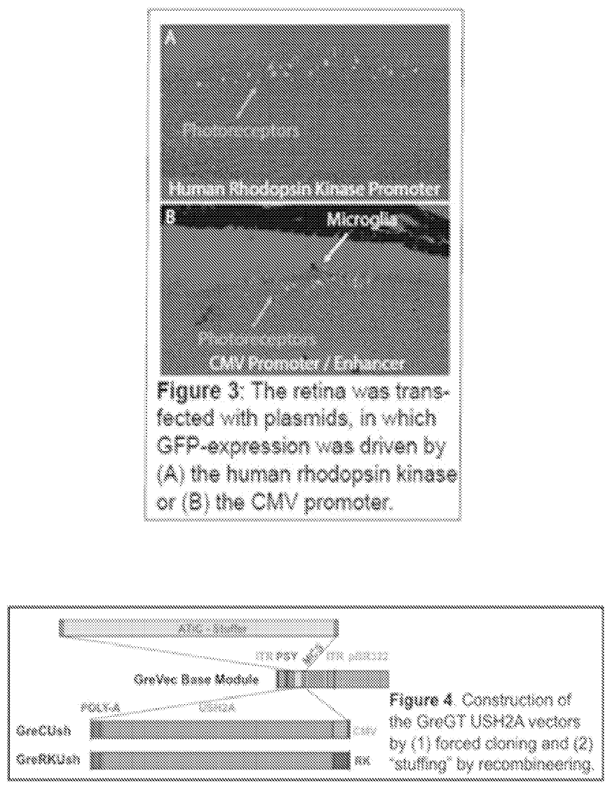

Promoters useful in the present disclosure will be integrated to study FGP expression. Once the activity of the RK promoter has been confirmed, its ability to drive expression of USH2A in photoreceptors will be investigated. The design of these vector genome molecules will follow that of the already produced CMV-GFP vector. The expression cassette will contain the specific RK promoter, the GFP sequences and the human growth hormone polyadenylation sites (HT-pA). It will be synthesized in its entirety together with the necessary cloning elements so that it can be easily moved into a viral vector, as shown in FIG. 4. The integrity of the assembled constructs will be confirmed by sequencing.

For purposes of further experiments/case studies, the vector genome modules will be packaged into human Ad5 (or other human serotypes) using standard co-transfection protocols.

To examine expression cassettes in transfection studies, three different test cells are used, HEK293, patient iPSC, and Y79 retinoblastoma cells for the in vitro studies. All experiments are preformed at least three times. HEK293 cells and undifferentiated patient iPSC cells that do not continually produce usherin, are well suited to study usherin protein production. To evaluate the activity of the RK promoter, these cells are cotransfected with plasmids encoding the Crx and Spl transcription factors. Transgene transcription will be measured by RT-qPCR using different sets of USH2A probes and protein production by immunostaining of fixed cells with a polyclonal anti-usherin antibody. The level of GFP expression is measured by FACS. The Y79 retinoblastoma cells constitutively express USH2A in a splice variant that skips exon 62. The expression of the vector-delivered transgene can be differentiated from constitutively transcribed USH2A with exon 62 specific RT-qPCR probes. No transcription factors have to be provided to examine RK promoter activity in Y79 cells. In a second step, the encapsidated vectors will be used to transduce the test cells at different multiplicities-of-infection. In previous studies, highest levels of transgene expression are observed after 48 hours. The percentage of transgene expression in tissue culture cells and the level of transgene expression per cell will be quantitatively analyzed by immunofluorescence in an FACS.

The studies are performed with GHFP and USH2A delivering vector genome module plasmids and packaged vectors. USH2 and autosomal recessive RP are autosomal recessive, but not X-linked, genetic diseases, and no sex-related phenotypical differences have been reported in humans or mice. Nevertheless, the C57BI/6 wild-type mice are used for GFP testing and Usha2a knockout (for USH2A testing) of both sexes. Four mice are included per group for molecular, biochemical and imaging studies, while 8 mice are included per group for electro-retinogram recording. A vector genome module devoid of a transgene will be used as a negative control.

Mice at one month age are injected with the encapsidated vectors by a subretinal injection procedure. A volume of ≤1 μl of vector suspensions of concentrations of 2×10⁹ IU/ml will be delivered.

The mice are sacrificed at day 21 post electroporation, when the photoreceptors just finish their differentiation, or at day 7 post transduction when transgene expression reaches their maximum levels and the injection-related wound is fully recovered. The whole mount retinas and retinal sections are analyzed for vector uptake. If effective transgene expression is seen, additional mice will be added to survey therapeutic effects and inflammation and toxicity responses at 3 and 6 months.

Besides in situ PCR with an ITR-specific probe to localize cells that have taken up the vector DNA after electroporation and transduction, the GFP test genes are directly visualized. The usherin expression in treated knockout mice will be detected by immunofluorescence and confocal microscopy. The number of transgene expression cells and the transgene expression intensity per cell will be scored using the ImageJ program. Additionally, GFP and USH2A expression in the retina will be evaluated by semi-quantitative immunoblotting analysis. In parallel, the retinal sections are examined by immune histology for evidence of inflammatory and immune responses. Usherin is essential for the Ush2 protein complex integrity in photoreceptors, we thus will examine the rescue of the USH2 protein complex in the treated Ush2a knockout retinas. Averages are taken from 4 sections per animal. Data from 4 mice per experimental and control groups (eye mock-injected or treated with control vectors) will be compared using Student's t-test and/or one-way ANOVA. Because Ush2a knockout mice do not show retinal degermation up to 1 year of age, we will assess the retinal health but not the rescue of retinal degeneration, in mice using electroretinogram (ERG) and optical coherence tomography (OCT) at 3 and 6 months after viral vector transduction. Based on the standard deviations of the previous ERG and OCT data, 8 mice will be tested per experimental and control groups. Student's t-tests and two-way ANOVA will be performed to analyze ERG and OCT data.

While multiple embodiments and related methods have been described in detail herein, it should be apparent that modifications and variations thereto are possible, all of which fall within the true spirit and scope of the invention. Further, since numerous modifications and changes will readily occur to those skilled in the art, it is not desired to limit the invention to the exact construction and operation shown and described, and accordingly, all suitable modifications and equivalents may be resorted to, falling within the scope of this disclosure.

1. An viral vector comprising a transgene construct, wherein the transgene construct is one of (a) a DNA sequence comprising a promoter element, a retinal gene's open reading frame being longer than 10 kb, and a terminator sequence, and (b) a DNA segment of retinal genes longer than 10 kb.

2. The viral vector of claim 1, wherein the vector is fully deleted of endogenous genes aside from ITR sequences.

3. The viral vector of claim 2, wherein the vector is deleted of enough endogenous adenoviral genes to make up to 10kb of space for the cargo sequence.

4. The viral vector of claim 1, wherein the viral vector is a fully or partially gutted mimivirus.

5. The viral vector of claim 1, wherein the viral vector is a fully or partially gutted adenovirus.

6. The viral vector of claim 5, wherein the viral vector is encapsidated in a capsid based on adenovirus type 5.

7. The viral vector of claim 5, wherein the viral vector is encapsidated in a capsid based on adenovirus type 6.

8. The viral vector of claim 5, wherein the viral vector is encapsidated in a capsid based on adenovirus and modified to remove RGD sequences from the capsid proteins.

9. The viral vector of claim 5, wherein the viral vector is encapsidated in a capsid based on adenovirus in families A-G.

10. The viral vector of claim 5, wherein the transgene construct is (a) a DNA sequence comprising a promoter element, a retinal gene's open reading frame being longer than 10 kb, and a terminator sequence.

11. The viral vector of claim 10, wherein the promoter element is a human rhodopsin kinase promoter.

12. The viral vector of claim 11 wherein the promoter element is a human rhodopsin kinase promoter.

13. The viral vector of claim 10, wherein the promoter element is a mouse rhodopsin kinase promoter.

14. The viral vector of claim 10, wherein the promoter element is a ubiquitous CMV promoter.

15. The viral vector of claim 10, wherein the promoter element is an upstream non-coding sequence of Usherin genome from nucleotides XXX to YYY.

16. The viral vector of claim 10, wherein the promoter element is a synthetic a transducin alpha-subunit promoter.

17. The viral vector of claim 10, wherein the promoter element is an inter-photoreceptor retinoid-binding protein promoter and a minimal sequence of the human transducin alpha-subunit promoter,

18. The viral vector of claim 10, wherein the promoter element is a chemically controlled tet inducible promoter.

19. The viral vector of claim 10, wherein the promoter element is a chemically inhibited promoter.

20. The viral vector of claim 10, wherein the promoter element is a human L-opsin promoter.

21. The viral vector of claim 10, wherein the promoter element is a human retinoschisin proximal promoter and the human interphotoreceptorretinoid-binding protein enhancer.

22. The viral vector of claim 10, wherein the promoter element is specific to photoreceptors.

23. The viral vector of claim 10, wherein the transgene construct is a nucleotide sequence which translates to Ush2A.

24. The viral vector of claim 10, wherein the transgene construct is a nucleotide sequence which translates to ADGV1.

25. The viral vector of claim 10, wherein the transgene construct is a nucleotide sequence which translates to eyes shut homolog.

26. The viral vector of claim 10, wherein the transgene construct is a nucleotide sequence which translates to Hem icentin.

27. The viral vector of claim 10, wherein the transgene construct is a nucleotide sequence which translates to Versican.

28. The viral vector of claim 5, wherein the transgene construct is (b) a DNA segment of retinal genes longer than 10 kb.

29. The viral vector of claim 28, wherein the DNA segment is at least a segment of the Ush2A gene.

30. The viral vector of claim 28, wherein the DNA segment is at least a segment of the ADGVI gene.

31. The viral vector of claim 28, wherein the DNA segment is at least a segment of the eyes shut homolog gene.

32. The viral vector of claim 28, wherein the DNA segment is at least a segment of the Hem icentin gene.

33. The viral vector of claim 28, wherein the DNA segment is at least a segment of the Versican gene.

34. The viral vector of claim 6, wherein the transgene construct is (b) a DNA segment of retinal genes longer than 10 kb.

36. (canceled)

37. (canceled)

38. (canceled)


SEQUENCE LISTING (3 sequences)

SEQ ID NO: 1 - DNA, 15612 bp, Homo sapiens
atgaattgtcctgtactgagcctcggtagcggctttttgttccaagtcatagaaatgctcatctttgcgtacttcgcgtcaatatctcttacagagtctcggggtctcttcccaaggcttgaaaatgtaggagcgtttaaaaaggtttccatcgtaccaacccaggctgtatgtggccttcctgaccgcagtactttttgccactcttccgccgctgctgagtcaattcagttttgcactcagcggttctgcattcaagattgcccctatcggtcttctcacccaacatatacggcactgttttctgcggggcttagttcctgtattacccctgacaagaatgatcttcaccctaacgcgcactcaaatagtgcatcatttatcttcggaaaccacaaatcctgtttttcttctcctccgtcccccaaactgatggcctcctttacgttggcggtttggcttaaaccggaacaacaaggagtaatgtgcgtgattgagaagacggttgatggacaaatcgtctttaagctgacgatatcagagaaagagacgatgttttattaccggacggtgaatgggctgcaacccccgattaaagtaatgactctcggtcggatcctcgtgaagaaatggattcacctgagtgtacaagtccatcaaactaagatttcattttttatcaacggagtcgagaaggaccataccccatttaacgctaggacattgagtgggagcattacagatttcgcctccggaacggtacagatcggccaatccttgaatggattggaacaattcgtcggaagaatgcaggacttccggctgtaccaggtagcactgacaaatcgagagatccttgaagtctttagtggggacctgttgcggctgcatgcccaatcacactgccggtgtccaggttctcacccgagggtacacccgctggctcagcggtactgtataccaaatgatgccggggacacagcagataacagagtcagtcgacttaacccggaggcacaccctctctcattcgtaaacgacaacgacgtggggacttcttgggtgtctaacgtcttcacaaatataacacaactgaatcaaggcgtcaccatctccgttgacctcgagaacgggcagtatcaggtgttctatattattatacaatttttcagtccacagcccacggagatacgaatccagcggaagaaagagaattccctggactgggaagactggcagtacttcgctcgcaattgcggggcgtttggcatgaagaataacggagacctggagaagcctgattccgtgaactgccttcagctctcaaacttcaccccctactcaagagggaatgtgacattctcaattcttactccgggccccaattatcggcccgggtacaacaacttttataacacgccttccctgcaagaatttgtaaaggcaacccaaatcagatttcacttccatggacaatactataccactgaaacagccgttaatctgaggcaccgctactacgccgtggatgaaatcacgatcagtggacggtgtcagtgccatggccacgctgataactgcgacacaactagccaaccatacagatgcctctgcagccaagaaagtttcactgagggccttcactgtgatagatgcctgcccctttataacgataagcctttcaggcaaggagaccaagtttatgcgtttaactgcaaaccatgccaatgcaacagccattccaagtcctgtcattacaacatttccgtcgacccttttccatttgagcattttaggggagggggcggagtatgtgatgactgcgaacataacactactggtagaaactgtgagttgtgcaaagattacttctttcgccaagtgggtgcggatccatccgcgatcgacgtatgtaagccctgtgactgtgacaccgtcgggacgcggaatggatctatcctttgtgatcaaataggtgggcaatgcaattgtaaacgacacgtatcaggtaggcagtgcaaccagtgtcaaaatggattctacaatctccaggaactcgaccctgacggatgtagtccatgcaactgtaacacctctggcaccgtagacggtgacataacatgtcatcagaatagtggacagtgtaaatgtaaggctaatgttatcgggctcaggtgcgatcattgcaatttcggttttaagttcctccgctcatttaatgatgtagggtgtgaaccgtgtcagtgtaaccttcacggtagtgttaacaagttttgtaacccccattctggtcagtgcgagtgcaagaaggaggccaaggggttgcaatgcgatacttgccgcgaaaatttttatgggcttgatgtaaccaactgtaaggcgtgcgactgcgatactgcaggatctctgccaggaactgtgtgcaacgcgaaaactggacagtgcatttgtaaaccgaatgtggaaggacgacagtgcaacaagtgcctggagggaaatttttatctccggcagaacaactcatttctctgtctcccatgtaattgcgataaaactggcaccattaatggcagcttgctttgtaataaaagcactggacaatgcccctgcaagctgggagttacggggctgcggtgtaaccaatgcgaaccacaccggtacaatctcaccatcgacaatttccaacattgtcagatgtgtgaatgcgattccctcgggacactccctggcacgatatgtgaccccattagtggccaatgcctgtgtgtacccaaccgacagggtcgacgatgtaatcagtgtcagccaggattttacataagcccgggaaacgccacaggttgcctgccctgctcatgtcacacgacgggagcagtcaatcacatatgcaatagcctcactgggcaatgcgtgtgccaagacgcaagcattgctggtcagcgctgtgatcagtgcaaagatcactacttcgggtttgacccacagacaggtcggtgccagccgtgcaactgccaccttagcggtgcattgaacgaaacatgtcatttggtaacgggtcagtgcttttgcaaacaatttgtaacgggttccaagtgtgacgcatgtgttccttctgccagccatcttgacgttaacaatctccttggttgttctaagactccatttcagcaaccaccgccaagaggccaggtccaaagctcatcagccatcaacctctcatggtcacctcccgattcaccgaacgcgcactggttgacctactcattgctccgcgatggattcgagatttataccaccgaggaccaatatccgtattccattcagtactttctggacactgacttgctgccctatactaaatatagttattatatcgagacgaccaatgtgcacggttctacgcgatctgtggccgtcacatataagactaagcctggagtgcctgaaggtaatctgactttgagctacataatcccaattggatccgacagcgtgacccttacgtggaccacgctttccaaccaatcaggaccaattgaaaaatacatcttgtcttgcgctccgttggcggggggtcaaccctgcgtctcatatgagggccatgagacaagtgcgactatatggaatctcgttccctttgcaaagtatgacttttctgtacaagcctgcacatctggcggatgcctgcatagtcttccaatcacagttaccacagcacaagcacccccccagaggttgagcccgccaaagatgcaaaagatttcttctacggaactccatgtggagtggagtcctccggcagaactcaatggtattatcattaggtatgaactgtacatgcgacggcttagatctaccaaagagactacgagtgaggagtcacgcgtgtttcagtcatctggctggttgtcaccccattcattcgtggagtctgcaaatgaaaacgcgttgaagcctcctcaaacaatgactacgataactggacttgagccatatactaaatatgagtttagagtactggctgtcaacatggccggctccgtctcaagtgcatgggtttccgagcgcacaggcgaatccgcgcctgtcttcatgataccccccagtgtttttcctctgtctagttactccctcaacatatcttgggaaaagcctgcggacaacgtgaccagaggtaaggtcgtgggctacgatatcaacatgctgtcagagcagtccccacagcagagcatcccgatggccttttcacaactcttgcatacggccaaatcacaagaactgtcatacacggtcgagggacttaaaccttatcggatctacgagtttacgatcacgctgtgcaactcagtcgggtgtgtcacttctgcgagcggtgcaggccagacgcttgcagcagcgccagcgcagctcaggccccctttggtcaagggaataaattctactacgatccatctccggtggtttcctccggaggaactgaatggccctagtcccatttatcagctcgaaaggagagagtcttcactgcctgcactcatgaccactatgatgaaaggaatccgctttattggaaacgggtactgcaaattccccagctccacgcaccctgtgaatacggattttacagggatcaaggcctcttttagaacgaaagtgccggagggcctcatcgttttcgctgctagccccggcaaccaagaggagtatttcgcccttcagttgaagaagggaaggctgtacttcctgttcgatcctcagggaagtccagttgaggttacgacaacaaatgaccatggtaaacaatacagcgatggtaaatggcacgagataatcgcgattagacaccaggcgtttgggcagatcacactcgacggcatttacactggatccagtgcaattctcaacggtagcacagttattggcgacaatacgggggtcttcttgggcggtctgccacgatcatacactatacttcgcaaggatcctgaaataatacagaagggttttgtcggatgccttaaggacgtgcattttatgaaaaactacaatccttccgctatatgggagccactggactggcagtcttcagaggaacaaatcaacgtgtataattcttgggaagggtgtcccgcgagccttaacgaaggggcacaatttttgggggcaggtttcctcgagttgcacccatacatgtttcatggggggatgaatttcgaaatttctttcaagtttcggacggaccaattgaacggcttgcttctgtttgtctacaacaaggacggccctgatttcctggccatggagctcaaaagtgggatactgaccttccgccttaatactagtctggcgtttacccaagtagatctgctgcttggcctttcttactgcaacgggaaatggaacaaagtgatcatcaaaaaagaaggctcctttatttctgcatctgtgaatggcctgatgaagcatgcctccgaaagtggtgaccaaccccttgtagtcaacagcccggtatatgttggggggatcccacaagaactcttgaacagttaccagcacttgtgcctcgaacaaggtttcggcggatgcatgaaggacgtgaagtttacccggggggccgtagtcaaccttgccagcgtatcatccggggccgtgcgggtcaatttggacggatgcctgtctacagattccgcagttaattgtcgagggaatgattcaatacttgtatatcaagggaaagaacaatcagtatacgaagggggcctccaaccttttacagagtatttgtatagagtcattgcaagtcatgaagggggatccgtttactcagactggtcacggggccgcacaaccggcgccgcgccacagtccgtccccacaccttccagggtgcggtcactgaacggttacagcatcgaagtgacgtgggatgaaccagtagtaaggggtgtgattgaaaagtatatattgaaagcttacagtgaggattccacacgccccccaaggatgccatccgcgtctgcggaattcgttaatacttccaaccttacaggcattttgacaggactgctcccttttaaaaattacgctgtcacacttactgcttgtaccctcgcggggtgcacagaaagtagtcatgcgctgaatatctccaccccgcaggaagcccctcaagaggttcagccgccggtcgcaaagagtcttccgagtagtctgctcctttcctggaacccccctaaaaaagcaaacggtatcatcacacaatactgcctctacatggacgggcggttgatttactctggtagtgaggaaaactacatagtaaccgatcttgctgtctttactccacatcaattcctcctttctgcgtgtactcatgtaggctgcacgaactcaagttgggtacttctctatactgcccagttgccacctgagcacgttgactctcccgtattgactgtattggacagcaggaccattcacattcaatggaagcagcctcggaaaataagcggcatccttgaaaggtatgttctctatatgtctaatcacacccatgacttcacaatttggagcgtcatttataatagtacagaactgtttcaggatcatatgttgcaatatgtactccctgggaataaatacttgataaagctgggtgcgtgcaccggaggtggttgtacggtgagcgaggcatctgaggcgcttacggacgaggacatcccagagggtgtacccgccccgaaggcccactcttacagccctgacagcttcaatgtgagctggactgaacccgaatacccaaatggcgtaataaccagttatggactgtacctcgatggtatattgatacataattcttccgaacttagttatagggcctacggatttgctccgtggagcttgcacagtttccgggtccaggcttgcaccgcgaaaggttgcgccctgggtccgctcgtggaaaatcgaacactcgaggccccacctgaggggaccgtgaatgtgtttgttaagacacagggaagtcgaaaagcgcatgtccgatgggaggccccatttagacctaatggcttgcttacgcactctgtgttgttcacggggatcttctacgtcgaccctgtaggtaataattacacattgctgaacgtcaccaaggtgatgtactccggagaggaaactaacctttgggtactgattgacggcctggtaccgtttacgaactatacagttcaagtgaacatcagtaacagccaagggtccctcattacggatccgattactatcgcgatgccgccgggtgctcctgatggagttctccccccccgcctttcatctgctactcctacttcattgcaggttgtgtggtcaacccccgcgagaaacaacgcgcctggtagccccagatatcaacttcaaatgcggagcggggattccacccacggtttcctcgagctcttttccaatccttcagcaagcttgtcctacgaagtttctgatctccagccatatactgaatacatgttccgactggtggcgagcaatggatttgggtcagcccacagttcttggatcccctttatgaccgctgaggacaagccagggcccgtggttccgcccatcctgctggatgtcaaaagtcgaatgatgctggtcacgtggcaacatccgagaaagtccaacggcgttataactcactacaacatttacctgcatggtcggctttacttgcgcaccccaggaaatgtaacgaactgtaccgttatgcacctccacccttataccgcctacaagttccaggtcgaggcatgtacgtccaaaggatgctcacttagccccgaaagccagaccgtttggacgcttcctggggctccggaagggatccctagtccggagctctttagcgatacacccaccagtgtgataatcagctggcagccccctactcatccgaatggtctcgtcgaaaatttcacgatagaaaggcgagttaaaggtaaagaggaagtgaccaccttggttactctgccccggagccatagtatgcgctttatagacaagacctccgcccttagcccctggaccaaatatgaataccgggttcttatgtccacgttgcatggagggacaaattccagcgcttgggtagaagtcactactcgaccaagccggcccgcgggcgttcaaccacctgttgttacggtactggagcctgatgctgtccaagttacgtggaaacccccactgatccagaatggtgacatactgagttacgagatacatatgccagaccctcacataacattgacgaacgttacctctgcagtgctttcccagaaagttactcacctcattccgtttacgaactatagtgtcacaatcgtggcctgtagcggtggtaacggatatcttggagggtgcaccgaaagcctccctacttacgtgacgacgcacccgacagttccgcagaatgtaggtcccctgtccgttattccgttgtccgaatcctatgtcgtgatatcttggcagcccccctctaaacctaatggacccaatctgaggtacgagctgcttcgacgcaagatccaacagccattggcgtccaacccgccggaagacctgaatcggtggcacaatatttatagtggcacgcaatggttgtacgaagacaagggcttgtccaggttcacgacctacgagtatatgcttttcgttcataattccgtcggatttaccccgtctcgcgaagtgaccgtcaccaccttggctggcttgccagaacgcggggcgaacctcactgcttcagtcctgaaccacaccgcgatagatgttcggtgggctaagccaactgtgcaagatctccagggcgaggttgaatactataccttgttttggtcctctgcaacgtccaatgatagtctcaagatattgccggatgttaacagtcacgtaattggacatctcaaacctaacacagaatactggatatttatttccgtgtttaatggagtgcactccataaacagtgctggactgcatgcgacgacttgtgatggtgagccacaggggatgcttccccctgaagtagttatcattaactcaaccgcagtcagagtaatttggacttctccaagcaacccaaatggagtagtaaccgagtactctatttatgtgaacaataaactgtacaagaccggcatgaacgtacccggctcatttattttgagagacctgtccccgtttacgatttacgatatacaggtggaggtctgtacaatatatgcatgcgtcaagtccaacggaacacaaatcactaccgtcgaagatacaccctcagatattcctacgccaaccattaggggtattacgtctaggagtcttcaaatagactgggtgtcaccccggaagcccaacgggattattctcggatatgacctgttgtggaagacttggtatccgtgcgccaaaacacagaaattggtccaagatcagtcagatgaattgtgtaaggcggtgcggtgtcaaaaacccgagagtatctgcggacacatctgttatagctcagaggcaaaagtatgctgtaacggtgttctgtacaatcccaaaccaggccatcgctgttgtgaggagaaatacatcccgttcgtgctgaactcaacgggggtctgttgcggtggtcgcattcaagaagcccagcccaaccaccagtgttgctctggttactacgcccgcatcctcccaggggaggtatgttgccctgacgaacaacacaacagggtaagcgtaggcattggcgactcctgttgtggccggatgccgtatagtactagcggtaaccaaatttgctgcgcgggaagacttcatgatggacacggacagaaatgttgcggccgccaaattgtatcaaatgacctcgaatgctgcggcggcgaagaaggcgtagtttacaataggctgccggggatgttctgttgcggacaagactacgtaaatatgagcgatactatttgctgttctgcaagctctggggagtcaaaggcccacataaaaaagaatgacccagtaccagttaagtgctgcgaaactgagctcattcccaagtcacagaaatgttgcaatggggtaggatataatccgttgaagtacgtctgctcagataagatttcaaccggtatgatgatgaaagagaccaaggaatgtagaattctgtgccccgccagtatggaagcaacggaacactgtggtcggtgcgactttaatttcacgagtcacatctgcactgtcatccgcggctcccataactcaactggcaaggcctctatagaggaaatgtgcagttccgcggaggaaaccatacatacgggaagcgtcaatacgtattcttatactgacgtcaacctgaagccctacatgacgtacgagtaccgaatcagcgcgtggaattcatacggccgcggattgagtaaagcagtcagggctagaacgaaggaggatgttccgcaaggcgtaagcccacccacttggacgaaaatagacaaccttgaggatacaattgtccttaattggaggaagccgatccagagtaacggtcctattatttactacattcttctgcggaatgggatagagaggtttcgaggcactagtctgagtttttccgataaggaaggtattcagccatttcaagaatacagctaccagctgaaagcatgcacagtggcgggatgtgccacctcaagcaaagtagtagccgcgactacccaaggcgttccggagagtatacttccgcccagtatcaccgcgctgagcgccgtagcattgcacctcagctggtcagttccggaaaaaagtaatggcgtcattaaggaatatcaaatacggcaggtgggaaaaggccttattcacactgatacgaccgaccgacggcaacatacagtgactggtctccaaccgtatacgaattattcattcacactgaccgcctgcacaagtgctggatgtacctctagtgagcccttccttgggcaaactctgcaagcggccccagaaggcgtttgggtcaccccacgccatattattatcaactctaccacggttgagctctactggtcccttccggagaaacccaacggcctggtatcccaatatcaactctccagaaatggtaacctgctctttttgggcggtagtgaggaacaaaatttcacggataagaatctcgagcctaactcaaggtatacttataaacttgaggtgaaaaccggaggcggcagttctgccagcgacgattatatagtacaaacacctatgtccacaccggaggaaatctatccaccgtataacataacagtgataggtccttactcaatatttgtagcgtggatcccgccagggatattgatacccgagattcctgtagagtataacgtgttgctcaatgatggctccgtgactccgcttgcattctcagttggacatcatcagagtacgctccttgaaaaccttacgccatttactcaatatgaaatccggatccaggcgtgccagaacggtagctgtggtgtgtcctctcggatgtttgtcaaaactcctgaagcggcaccaatggacctcaacagtcccgtgcttaaggctttgggctcagcttgcatagaaatcaagtggatgccacctgagaaaccaaatggtatcataattaactatttcatttaccggcgccccgcagggatcgaggaggaaagcgtgctttttgtatggtctgaaggggcactggaattcatggacgaaggagataccttgaggccatttaccctttacgagtaccgcgtgagagcttgcaactctaaggggagcgtcgaaagcttgtggtctctcacgcagacccttgaggctcctccccaagatttcccagcgccgtgggcccaggcgacatctgcacacagcgtgttgcttaactggactaaacctgaaagcccgaacggcatcatctcacattatagggttgtttatcaagaacggccggacgatccgactttcaattctcccacggtgcacgcctttacagtaaagggtactagccatcaggcccacctctacggtctcgaaccattcacgacgtatcgcatcggcgttgtagcggccaatcatgctggtgagatacttagtccgtggacgctgatacaaactctggagtctagcccctccggtttgcgcaacttcattgtggagcaaaaagagaatggtcgagctcttctccttcaatggtccgaacccatgcgcacgaatggcgtgataaagacttataatatattctcagatggcttccttgaatactcaggtttgaaccgacagttcctttttaggaggcttgacccattcactctctacacgctgacgctcgaagcgtgtaccagggccggttgtgcacattctgcaccacagcccctttggacggatgaggctccccctgattctcaactcgccccaacggtgcatagcgtcaagtcaacctcagtagagcttagttggtctgagccagtgaaccctaatggaaaaattataagatacgaagtgatacgccgatgtttcgaaggtaaagcttggggcaatcaaacaatccaggccgatgagaaaattgtatttacagaatataacaccgagaggaacacgttcatgtataacgataccgggctgcaaccttggactcaatgtgaatataaaatttatacttggaattcagcgggccatacctgctcctcatggaacgtggtgcggactctccaagcaccgccggagggcctttcaccgcctgtaatctcttacgtttccatgaatcctcagaagctgcttatctcctggataccacccgaacaaagcaacggaatcatacagtcttatcggttgcagaggaacgagatgttgtatcctttttccttcgaccccgtcacttttaattatacagacgaggaactgttgccatttagcacctattcctacgcccttcaagcgtgtacgagcggtggctgctccacatccaaaccgaccagcattaccacacttgaagccgctccaagcgaggtctctccacctgacctctgggccgtttctgctacgcaaatgaatgtgtgctggtccccgcccacggttcagaatgggaaaatcactaaatacctcgtaaggtatgacaataaagaaagtctggctggacaagggctctgccttctggtcagtcacctgcagccttatagtcaatataattttagtctcgtagcgtgtaccaacggcggctgtacagcctctgtttcaaaaagcgcttggacaatggaggccttgcccgagaacatggactctccgaccctgcaggtgaccggatcagaatccatagaaatcacgtggaagccaccacgcaatccgaacggacagattcgaagctacgaattgcgccgggatgggactatcgtgtacacagggctggagactcgatacagggacttcaccctgactccgggagtggaatattcatatactgtcacggccagtaactcacagggtggaatcctgagtcctctggtgaaagatcgcactagtcccagtgcgccctctggcatggagccaccgaaattgcaagcaagagggccccaggagatcctggtcaattgggatcctcccgttaggactaacggtgacattattaactacaccttgtttatcagggagttgttcgaaagggaaaccaagattatccacattaatactacccataacagcttcggcatgcagtcatatattgtaaaccaattgaaaccgttccaccgatacgagattaggatccaggcatgcacgaccttggggtgtgcatccagcgactggacgtttattcaaacgccagaaattgcgcctttgatgcagccgcccccgcacctggaggtacagatggctccgggcgggttccaacccacagtctctcttctctggaccggacctctgcagcctaatggaaaggtcctgtattatgaactttacagacgacagatcgccactcaaccccgcaaatccaatcctgtgcttatctacaacgggtctagcacgagcttcattgatagcgaattgctgccttttaccgagtacgagtaccaagtgtgggcagtgaactcagcggggaaagcgccttcctcttggacgtggtgccggacgggacctgcgccacctgaggggctgcgggcacccacgttccatgttatcagctcaacgcaagctgtcgtaaatatctccgctccaggtaaaccgaacggtattgtatccctctataggttgttctcttcctcagcacacggcgctgaaacagtactgagcgaaggaatggcaacccagcagaccctgcacgggttgcaggcctttacgaactatagcattggagtcgaagcgtgcacctgcttcaactgttgtagcaaaggccccaccgcggaactccggacccatcctgctccaccctctggcctgtcctccccacaaatagggaccttggcctctcgcacagcgagtttccgctggagtccgccgatgtttccaaatggggtcatacattcatacgagcttcagttccatgtcgcgtgcccccctgactctgcgctgccttgcaccccaagccaaatcgagactaagtacacagggctgggccagaaagccagtcttgggggtctgcaaccctatacgacgtataagcttagggtagtcgcccacaacgaggtaggtagtacggccagtgaatggataagtttcacgacacaaaaagagttgccacagtaccgggccccgttctccgtggacagcaacctcagtgttgtatgtgtcaattggtctgatacattccttctgaatgggcaacttaaggaatatgttttgacagacggggggagacgcgtttactctgggcttgacacgactctgtatattccacggaccgcggataagacttttttttttcaggtgatttgcactactgacgaggggtctgttaaaaccccgttgatacagtacgatacctcaacgggattgggtctcgttcttacaaccccggggaagaaaaagggcagcaggagcaaatctacagagttctatagtgagctgtggtttattgtcctgatggccatgttgggtctcattcttttggcgatatttctgtcactcatattgcaaagaaagatacataaggagccttatatcagagagcgccctcctttggttcctttgcaaaagagaatgtcaccgcttaacgtttatccgcctggtgagaatcacatgggactggctgataccaagatccctaggtctggtacacctgtgtctatccgcagtaaccgctccgcttgtgtactgcgaataccgtcacagaatcagacaagtctgacatacagccaaggcagcctccaccggagtgtatctcaactcatggacattcaggataagaaggtcctcatggacaacagcttgtgggaagcgatcatgggtcacaactctggcctctatgtagacgaggaagacctcatgaatgcaataaaggatttttcaagcgtgactaaagaacggacgactttcacggacacgcacttgtaataa

SEQ ID NO: 2 - DNA, 35289 bp, Homo sapiens
tcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattacgggcccatatagtttaaacatacatcatcaataatataccttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatggctgccgctatgcccggggggaggcctaatcggccttagcgatcgcgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggcgcgccagccgccaccatgaattgtcctgtactgagcctcggtagcggctttttgttccaagtcatagaaatgctcatctttgcgtacttcgcgtcaatatctcttacagagtctcggggtctcttcccaaggcttgaaaatgtaggagcgtttaaaaaggtttccatcgtaccaacccaggctgtatgtggccttcctgaccgcagtactttttgccactcttccgccgctgctgagtcaattcagttttgcactcagcggttctgcattcaagattgcccctatcggtcttctcacccaacatatacggcactgttttctgcggggcttagttcctgtattacccctgacaagaatgatcttcaccctaacgcgcactcaaatagtgcatcatttatcttcggaaaccacaaatcctgtttttcttctcctccgtcccccaaactgatggcctcctttacgttggcggtttggcttaaaccggaacaacaaggagtaatgtgcgtgattgagaagacggttgatggacaaatcgtctttaagctgacgatatcagagaaagagacgatgttttattaccggacggtgaatgggctgcaacccccgattaaagtaatgactctcggtcggatcctcgtgaagaaatggattcacctgagtgtacaagtccatcaaactaagatttcattttttatcaacggagtcgagaaggaccataccccatttaacgctaggacattgagtgggagcattacagatttcgcctccggaacggtacagatcggccaatccttgaatggattggaacaattcgtcggaagaatgcaggacttccggctgtaccaggtagcactgacaaatcgagagatccttgaagtctttagtggggacctgttgcggctgcatgcccaatcacactgccggtgtccaggttctcacccgagggtacacccgctggctcagcggtactgtataccaaatgatgccggggacacagcagataacagagtcagtcgacttaacccggaggcacaccctctctcattcgtaaacgacaacgacgtggggacttcttgggtgtctaacgtcttcacaaatataacacaactgaatcaaggcgtcaccatctccgttgacctcgagaacgggcagtatcaggtgttctatattattatacaatttttcagtccacagcccacggagatacgaatccagcggaagaaagagaattccctggactgggaagactggcagtacttcgctcgcaattgcggggcgtttggcatgaagaataacggagacctggagaagcctgattccgtgaactgccttcagctctcaaacttcaccccctactcaagagggaatgtgacattctcaattcttactccgggccccaattatcggcccgggtacaacaacttttataacacgccttccctgcaagaatttgtaaaggcaacccaaatcagatttcacttccatggacaatactataccactgaaacagccgttaatctgaggcaccgctactacgccgtggatgaaatcacgatcagtggacggtgtcagtgccatggccacgctgataactgcgacacaactagccaaccatacagatgcctctgcagccaagaaagtttcactgagggccttcactgtgatagatgcctgcccctttataacgataagcctttcaggcaaggagaccaagtttatgcgtttaactgcaaaccatgccaatgcaacagccattccaagtcctgtcattacaacatttccgtcgacccttttccatttgagcattttaggggagggggcggagtatgtgatgactgcgaacataacactactggtagaaactgtgagttgtgcaaagattacttctttcgccaagtgggtgcggatccatccgcgatcgacgtatgtaagccctgtgactgtgacaccgtcgggacgcggaatggatctatcctttgtgatcaaataggtgggcaatgcaattgtaaacgacacgtatcaggtaggcagtgcaaccagtgtcaaaatggattctacaatctccaggaactcgaccctgacggatgtagtccatgcaactgtaacacctctggcaccgtagacggtgacataacatgtcatcagaatagtggacagtgtaaatgtaaggctaatgttatcgggctcaggtgcgatcattgcaatttcggttttaagttcctccgctcatttaatgatgtagggtgtgaaccgtgtcagtgtaaccttcacggtagtgttaacaagttttgtaacccccattctggtcagtgcgagtgcaagaaggaggccaaggggttgcaatgcgatacttgccgcgaaaatttttatgggcttgatgtaaccaactgtaaggcgtgcgactgcgatactgcaggatctctgccaggaactgtgtgcaacgcgaaaactggacagtgcatttgtaaaccgaatgtggaaggacgacagtgcaacaagtgcctggagggaaatttttatctccggcagaacaactcatttctctgtctcccatgtaattgcgataaaactggcaccattaatggcagcttgctttgtaataaaagcactggacaatgcccctgcaagctgggagttacggggctgcggtgtaaccaatgcgaaccacaccggtacaatctcaccatcgacaatttccaacattgtcagatgtgtgaatgcgattccctcgggacactccctggcacgatatgtgaccccattagtggccaatgcctgtgtgtacccaaccgacagggtcgacgatgtaatcagtgtcagccaggattttacataagcccgggaaacgccacaggttgcctgccctgctcatgtcacacgacgggagcagtcaatcacatatgcaatagcctcactgggcaatgcgtgtgccaagacgcaagcattgctggtcagcgctgtgatcagtgcaaagatcactacttcgggtttgacccacagacaggtcggtgccagccgtgcaactgccaccttagcggtgcattgaacgaaacatgtcatttggtaacgggtcagtgcttttgcaaacaatttgtaacgggttccaagtgtgacgcatgtgttccttctgccagccatcttgacgttaacaatctccttggttgttctaagactccatttcagcaaccaccgccaagaggccaggtccaaagctcatcagccatcaacctctcatggtcacctcccgattcaccgaacgcgcactggttgacctactcattgctccgcgatggattcgagatttataccaccgaggaccaatatccgtattccattcagtactttctggacactgacttgctgccctatactaaatatagttattatatcgagacgaccaatgtgcacggttctacgcgatctgtggccgtcacatataagactaagcctggagtgcctgaaggtaatctgactttgagctacataatcccaattggatccgacagcgtgacccttacgtggaccacgctttccaaccaatcaggaccaattgaaaaatacatcttgtcttgcgctccgttggcggggggtcaaccctgcgtctcatatgagggccatgagacaagtgcgactatatggaatctcgttccctttgcaaagtatgacttttctgtacaagcctgcacatctggcggatgcctgcatagtcttccaatcacagttaccacagcacaagcacccccccagaggttgagcccgccaaagatgcaaaagatttcttctacggaactccatgtggagtggagtcctccggcagaactcaatggtattatcattaggtatgaactgtacatgcgacggcttagatctaccaaagagactacgagtgaggagtcacgcgtgtttcagtcatctggctggttgtcaccccattcattcgtggagtctgcaaatgaaaacgcgttgaagcctcctcaaacaatgactacgataactggacttgagccatatactaaatatgagtttagagtactggctgtcaacatggccggctccgtctcaagtgcatgggtttccgagcgcacaggcgaatccgcgcctgtcttcatgataccccccagtgtttttcctctgtctagttactccctcaacatatcttgggaaaagcctgcggacaacgtgaccagaggtaaggtcgtgggctacgatatcaacatgctgtcagagcagtccccacagcagagcatcccgatggccttttcacaactcttgcatacggccaaatcacaagaactgtcatacacggtcgagggacttaaaccttatcggatctacgagtttacgatcacgctgtgcaactcagtcgggtgtgtcacttctgcgagcggtgcaggccagacgcttgcagcagcgccagcgcagctcaggccccctttggtcaagggaataaattctactacgatccatctccggtggtttcctccggaggaactgaatggccctagtcccatttatcagctcgaaaggagagagtcttcactgcctgcactcatgaccactatgatgaaaggaatccgctttattggaaacgggtactgcaaattccccagctccacgcaccctgtgaatacggattttacagggatcaaggcctcttttagaacgaaagtgccggagggcctcatcgttttcgctgctagccccggcaaccaagaggagtatttcgcccttcagttgaagaagggaaggctgtacttcctgttcgatcctcagggaagtccagttgaggttacgacaacaaatgaccatggtaaacaatacagcgatggtaaatggcacgagataatcgcgattagacaccaggcgtttgggcagatcacactcgacggcatttacactggatccagtgcaattctcaacggtagcacagttattggcgacaatacgggggtcttcttgggcggtctgccacgatcatacactatacttcgcaaggatcctgaaataatacagaagggttttgtcggatgccttaaggacgtgcattttatgaaaaactacaatccttccgctatatgggagccactggactggcagtcttcagaggaacaaatcaacgtgtataattcttgggaagggtgtcccgcgagccttaacgaaggggcacaatttttgggggcaggtttcctcgagttgcacccatacatgtttcatggggggatgaatttcgaaatttctttcaagtttcggacggaccaattgaacggcttgcttctgtttgtctacaacaaggacggccctgatttcctggccatggagctcaaaagtgggatactgaccttccgccttaatactagtctggcgtttacccaagtagatctgctgcttggcctttcttactgcaacgggaaatggaacaaagtgatcatcaaaaaagaaggctcctttatttctgcatctgtgaatggcctgatgaagcatgcctccgaaagtggtgaccaaccccttgtagtcaacagcccggtatatgttggggggatcccacaagaactcttgaacagttaccagcacttgtgcctcgaacaaggtttcggcggatgcatgaaggacgtgaagtttacccggggggccgtagtcaaccttgccagcgtatcatccggggccgtgcgggtcaatttggacggatgcctgtctacagattccgcagttaattgtcgagggaatgattcaatacttgtatatcaagggaaagaacaatcagtatacgaagggggcctccaaccttttacagagtatttgtatagagtcattgcaagtcatgaagggggatccgtttactcagactggtcacggggccgcacaaccggcgccgcgccacagtccgtccccacaccttccagggtgcggtcactgaacggttacagcatcgaagtgacgtgggatgaaccagtagtaaggggtgtgattgaaaagtatatattgaaagcttacagtgaggattccacacgccccccaaggatgccatccgcgtctgcggaattcgttaatacttccaaccttacaggcattttgacaggactgctcccttttaaaaattacgctgtcacacttactgcttgtaccctcgcggggtgcacagaaagtagtcatgcgctgaatatctccaccccgcaggaagcccctcaagaggttcagccgccggtcgcaaagagtcttccgagtagtctgctcctttcctggaacccccctaaaaaagcaaacggtatcatcacacaatactgcctctacatggacgggcggttgatttactctggtagtgaggaaaactacatagtaaccgatcttgctgtctttactccacatcaattcctcctttctgcgtgtactcatgtaggctgcacgaactcaagttgggtacttctctatactgcccagttgccacctgagcacgttgactctcccgtattgactgtattggacagcaggaccattcacattcaatggaagcagcctcggaaaataagcggcatccttgaaaggtatgttctctatatgtctaatcacacccatgacttcacaatttggagcgtcatttataatagtacagaactgtttcaggatcatatgttgcaatatgtactccctgggaataaatacttgataaagctgggtgcgtgcaccggaggtggttgtacggtgagcgaggcatctgaggcgcttacggacgaggacatcccagagggtgtacccgccccgaaggcccactcttacagccctgacagcttcaatgtgagctggactgaacccgaatacccaaatggcgtaataaccagttatggactgtacctcgatggtatattgatacataattcttccgaacttagttatagggcctacggatttgctccgtggagcttgcacagtttccgggtccaggcttgcaccgcgaaaggttgcgccctgggtccgctcgtggaaaatcgaacactcgaggccccacctgaggggaccgtgaatgtgtttgttaagacacagggaagtcgaaaagcgcatgtccgatgggaggccccatttagacctaatggcttgcttacgcactctgtgttgttcacggggatcttctacgtcgaccctgtaggtaataattacacattgctgaacgtcaccaaggtgatgtactccggagaggaaactaacctttgggtactgattgacggcctggtaccgtttacgaactatacagttcaagtgaacatcagtaacagccaagggtccctcattacggatccgattactatcgcgatgccgccgggtgctcctgatggagttctccccccccgcctttcatctgctactcctacttcattgcaggttgtgtggtcaacccccgcgagaaacaacgcgcctggtagccccagatatcaacttcaaatgcggagcggggattccacccacggtttcctcgagctcttttccaatccttcagcaagcttgtcctacgaagtttctgatctccagccatatactgaatacatgttccgactggtggcgagcaatggatttgggtcagcccacagttcttggatcccctttatgaccgctgaggacaagccagggcccgtggttccgcccatcctgctggatgtcaaaagtcgaatgatgctggtcacgtggcaacatccgagaaagtccaacggcgttataactcactacaacatttacctgcatggtcggctttacttgcgcaccccaggaaatgtaacgaactgtaccgttatgcacctccacccttataccgcctacaagttccaggtcgaggcatgtacgtccaaaggatgctcacttagccccgaaagccagaccgtttggacgcttcctggggctccggaagggatccctagtccggagctctttagcgatacacccaccagtgtgataatcagctggcagccccctactcatccgaatggtctcgtcgaaaatttcacgatagaaaggcgagttaaaggtaaagaggaagtgaccaccttggttactctgccccggagccatagtatgcgctttatagacaagacctccgcccttagcccctggaccaaatatgaataccgggttcttatgtccacgttgcatggagggacaaattccagcgcttgggtagaagtcactactcgaccaagccggcccgcgggcgttcaaccacctgttgttacggtactggagcctgatgctgtccaagttacgtggaaacccccactgatccagaatggtgacatactgagttacgagatacatatgccagaccctcacataacattgacgaacgttacctctgcagtgctttcccagaaagttactcacctcattccgtttacgaactatagtgtcacaatcgtggcctgtagcggtggtaacggatatcttggagggtgcaccgaaagcctccctacttacgtgacgacgcacccgacagttccgcagaatgtaggtcccctgtccgttattccgttgtccgaatcctatgtcgtgatatcttggcagcccccctctaaacctaatggacccaatctgaggtacgagctgcttcgacgcaagatccaacagccattggcgtccaacccgccggaagacctgaatcggtggcacaatatttatagtggcacgcaatggttgtacgaagacaagggcttgtccaggttcacgacctacgagtatatgcttttcgttcataattccgtcggatttaccccgtctcgcgaagtgaccgtcaccaccttggctggcttgccagaacgcggggcgaacctcactgcttcagtcctgaaccacaccgcgatagatgttcggtgggctaagccaactgtgcaagatctccagggcgaggttgaatactataccttgttttggtcctctgcaacgtccaatgatagtctcaagatattgccggatgttaacagtcacgtaattggacatctcaaacctaacacagaatactggatatttatttccgtgtttaatggagtgcactccataaacagtgctggactgcatgcgacgacttgtgatggtgagccacaggggatgcttccccctgaagtagttatcattaactcaaccgcagtcagagtaatttggacttctccaagcaacccaaatggagtagtaaccgagtactctatttatgtgaacaataaactgtacaagaccggcatgaacgtacccggctcatttattttgagagacctgtccccgtttacgatttacgatatacaggtggaggtctgtacaatatatgcatgcgtcaagtccaacggaacacaaatcactaccgtcgaagatacaccctcagatattcctacgccaaccattaggggtattacgtctaggagtcttcaaatagactgggtgtcaccccggaagcccaacgggattattctcggatatgacctgttgtggaagacttggtatccgtgcgccaaaacacagaaattggtccaagatcagtcagatgaattgtgtaaggcggtgcggtgtcaaaaacccgagagtatctgcggacacatctgttatagctcagaggcaaaagtatgctgtaacggtgttctgtacaatcccaaaccaggccatcgctgttgtgaggagaaatacatcccgttcgtgctgaactcaacgggggtctgttgcggtggtcgcattcaagaagcccagcccaaccaccagtgttgctctggttactacgcccgcatcctcccaggggaggtatgttgccctgacgaacaacacaacagggtaagcgtaggcattggcgactcctgttgtggccggatgccgtatagtactagcggtaaccaaatttgctgcgcgggaagacttcatgatggacacggacagaaatgttgcggccgccaaattgtatcaaatgacctcgaatgctgcggcggcgaagaaggcgtagtttacaataggctgccggggatgttctgttgcggacaagactacgtaaatatgagcgatactatttgctgttctgcaagctctggggagtcaaaggcccacataaaaaagaatgacccagtaccagttaagtgctgcgaaactgagctcattcccaagtcacagaaatgttgcaatggggtaggatataatccgttgaagtacgtctgctcagataagatttcaaccggtatgatgatgaaagagaccaaggaatgtagaattctgtgccccgccagtatggaagcaacggaacactgtggtcggtgcgactttaatttcacgagtcacatctgcactgtcatccgcggctcccataactcaactggcaaggcctctatagaggaaatgtgcagttccgcggaggaaaccatacatacgggaagcgtcaatacgtattcttatactgacgtcaacctgaagccctacatgacgtacgagtaccgaatcagcgcgtggaattcatacggccgcggattgagtaaagcagtcagggctagaacgaaggaggatgttccgcaaggcgtaagcccacccacttggacgaaaatagacaaccttgaggatacaattgtccttaattggaggaagccgatccagagtaacggtcctattatttactacattcttctgcggaatgggatagagaggtttcgaggcactagtctgagtttttccgataaggaaggtattcagccatttcaagaatacagctaccagctgaaagcatgcacagtggcgggatgtgccacctcaagcaaagtagtagccgcgactacccaaggcgttccggagagtatacttccgcccagtatcaccgcgctgagcgccgtagcattgcacctcagctggtcagttccggaaaaaagtaatggcgtcattaaggaatatcaaatacggcaggtgggaaaaggccttattcacactgatacgaccgaccgacggcaacatacagtgactggtctccaaccgtatacgaattattcattcacactgaccgcctgcacaagtgctggatgtacctctagtgagcccttccttgggcaaactctgcaagcggccccagaaggcgtttgggtcaccccacgccatattattatcaactctaccacggttgagctctactggtcccttccggagaaacccaacggcctggtatcccaatatcaactctccagaaatggtaacctgctctttttgggcggtagtgaggaacaaaatttcacggataagaatctcgagcctaactcaaggtatacttataaacttgaggtgaaaaccggaggcggcagttctgccagcgacgattatatagtacaaacacctatgtccacaccggaggaaatctatccaccgtataacataacagtgataggtccttactcaatatttgtagcgtggatcccgccagggatattgatacccgagattcctgtagagtataacgtgttgctcaatgatggctccgtgactccgcttgcattctcagttggacatcatcagagtacgctccttgaaaaccttacgccatttactcaatatgaaatccggatccaggcgtgccagaacggtagctgtggtgtgtcctctcggatgtttgtcaaaactcctgaagcggcaccaatggacctcaacagtcccgtgcttaaggctttgggctcagcttgcatagaaatcaagtggatgccacctgagaaaccaaatggtatcataattaactatttcatttaccggcgccccgcagggatcgaggaggaaagcgtgctttttgtatggtctgaaggggcactggaattcatggacgaaggagataccttgaggccatttaccctttacgagtaccgcgtgagagcttgcaactctaaggggagcgtcgaaagcttgtggtctctcacgcagacccttgaggctcctccccaagatttcccagcgccgtgggcccaggcgacatctgcacacagcgtgttgcttaactggactaaacctgaaagcccgaacggcatcatctcacattatagggttgtttatcaagaacggccggacgatccgactttcaattctcccacggtgcacgcctttacagtaaagggtactagccatcaggcccacctctacggtctcgaaccattcacgacgtatcgcatcggcgttgtagcggccaatcatgctggtgagatacttagtccgtggacgctgatacaaactctggagtctagcccctccggtttgcgcaacttcattgtggagcaaaaagagaatggtcgagctcttctccttcaatggtccgaacccatgcgcacgaatggcgtgataaagacttataatatattctcagatggcttccttgaatactcaggtttgaaccgacagttcctttttaggaggcttgacccattcactctctacacgctgacgctcgaagcgtgtaccagggccggttgtgcacattctgcaccacagcccctttggacggatgaggctccccctgattctcaactcgccccaacggtgcatagcgtcaagtcaacctcagtagagcttagttggtctgagccagtgaaccctaatggaaaaattataagatacgaagtgatacgccgatgtttcgaaggtaaagcttggggcaatcaaacaatccaggccgatgagaaaattgtatttacagaatataacaccgagaggaacacgttcatgtataacgataccgggctgcaaccttggactcaatgtgaatataaaatttatacttggaattcagcgggccatacctgctcctcatggaacgtggtgcggactctccaagcaccgccggagggcctttcaccgcctgtaatctcttacgtttccatgaatcctcagaagctgcttatctcctggataccacccgaacaaagcaacggaatcatacagtcttatcggttgcagaggaacgagatgttgtatcctttttccttcgaccccgtcacttttaattatacagacgaggaactgttgccatttagcacctattcctacgcccttcaagcgtgtacgagcggtggctgctccacatccaaaccgaccagcattaccacacttgaagccgctccaagcgaggtctctccacctgacctctgggccgtttctgctacgcaaatgaatgtgtgctggtccccgcccacggttcagaatgggaaaatcactaaatacctcgtaaggtatgacaataaagaaagtctggctggacaagggctctgccttctggtcagtcacctgcagccttatagtcaatataattttagtctcgtagcgtgtaccaacggcggctgtacagcctctgtttcaaaaagcgcttggacaatggaggccttgcccgagaacatggactctccgaccctgcaggtgaccggatcagaatccatagaaatcacgtggaagccaccacgcaatccgaacggacagattcgaagctacgaattgcgccgggatgggactatcgtgtacacagggctggagactcgatacagggacttcaccctgactccgggagtggaatattcatatactgtcacggccagtaactcacagggtggaatcctgagtcctctggtgaaagatcgcactagtcccagtgcgccctctggcatggagccaccgaaattgcaagcaagagggccccaggagatcctggtcaattgggatcctcccgttaggactaacggtgacattattaactacaccttgtttatcagggagttgttcgaaagggaaaccaagattatccacattaatactacccataacagcttcggcatgcagtcatatattgtaaaccaattgaaaccgttccaccgatacgagattaggatccaggcatgcacgaccttggggtgtgcatccagcgactggacgtttattcaaacgccagaaattgcgcctttgatgcagccgcccccgcacctggaggtacagatggctccgggcgggttccaacccacagtctctcttctctggaccggacctctgcagcctaatggaaaggtcctgtattatgaactttacagacgacagatcgccactcaaccccgcaaatccaatcctgtgcttatctacaacgggtctagcacgagcttcattgatagcgaattgctgccttttaccgagtacgagtaccaagtgtgggcagtgaactcagcggggaaagcgccttcctcttggacgtggtgccggacgggacctgcgccacctgaggggctgcgggcacccacgttccatgttatcagctcaacgcaagctgtcgtaaatatctccgctccaggtaaaccgaacggtattgtatccctctataggttgttctcttcctcagcacacggcgctgaaacagtactgagcgaaggaatggcaacccagcagaccctgcacgggttgcaggcctttacgaactatagcattggagtcgaagcgtgcacctgcttcaactgttgtagcaaaggccccaccgcggaactccggacccatcctgctccaccctctggcctgtcctccccacaaatagggaccttggcctctcgcacagcgagtttccgctggagtccgccgatgtttccaaatggggtcatacattcatacgagcttcagttccatgtcgcgtgcccccctgactctgcgctgccttgcaccccaagccaaatcgagactaagtacacagggctgggccagaaagccagtcttgggggtctgcaaccctatacgacgtataagcttagggtagtcgcccacaacgaggtaggtagtacggccagtgaatggataagtttcacgacacaaaaagagttgccacagtaccgggccccgttctccgtggacagcaacctcagtgttgtatgtgtcaattggtctgatacattccttctgaatgggcaacttaaggaatatgttttgacagacggggggagacgcgtttactctgggcttgacacgactctgtatattccacggaccgcggataagacttttttttttcaggtgatttgcactactgacgaggggtctgttaaaaccccgttgatacagtacgatacctcaacgggattgggtctcgttcttacaaccccggggaagaaaaagggcagcaggagcaaatctacagagttctatagtgagctgtggtttattgtcctgatggccatgttgggtctcattcttttggcgatatttctgtcactcatattgcaaagaaagatacataaggagccttatatcagagagcgccctcctttggttcctttgcaaaagagaatgtcaccgcttaacgtttatccgcctggtgagaatcacatgggactggctgataccaagatccctaggtctggtacacctgtgtctatccgcagtaaccgctccgcttgtgtactgcgaataccgtcacagaatcagacaagtctgacatacagccaaggcagcctccaccggagtgtatctcaactcatggacattcaggataagaaggtcctcatggacaacagcttgtgggaagcgatcatgggtcacaactctggcctctatgtagacgaggaagacctcatgaatgcaataaaggatttttcaagcgtgactaaagaacggacgactttcacggacacgcacttgtaataaggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactggccggccatcgatgtaagaaattatattacaagctaaagcaacagctgtgggtggaagaagagaagaaataatgtgttttttctttcttctttgatggcaaactcaacagactctgcctaaagcagaactgtagttaaaaggagactgcaacttcagagttttccattacctagtttctcaacactggcagatttttttaggagctagtatctcttgtgaataaacataagaagttcagcacacagaaagaaatggtgggccagcactaattaaaaacaaaaatgagccgggtacaatggctcacccccatgaccccagcactttgggaggccgaggtgggtggatcacaaggtcaggagttcgagatcagcctggtcaacatggtgaaacccccgtctctactaaaaacacaaaaattagctgggtgtggtggcgggcgcatgtaatcccagctactagggaggctgaggcaggagaatcgcttgaacctggaaggtggaggttgtagtgagccgaaaccgcgccactgcactccagcctggccaacaagagcgagactccgtctcaaaaaaaaaaaaaaaaaacccaaaaaacaaaaatgtgtaatttcatgtccttcatgggttaagatagtttttaaagaccttgccaaatatttactgagttgcctactacgatgttaagcactgttctaggtattcaggatacatcagtgaaaaaaaccgtccctgcaaccctcttaaaaaaaagtcactgtccttatggagcttactggggaggcaaataaaaaataggcaataaatagtatacaataaaagaaaatattaggtatttatcaaaatgagttgaaaacttgcacacaaatgattatagcaattttgttcataaactaccaaaaactgtccttgaagttttgatgcccttcaatatgtccatggataaactgtggtacattcatacacagtgaaatattattcagcattaagaaataagctaccaacccacaaaaagatgtggaagaaacttaatgcatgttgttaactgaaagtgcctaaaagggctccatactgtatgatcccaactatctgacattctggaaatgggtacagagtcagtaaaaacagctgggcatggtggtgcatgcctgtaatcccagctacttgagaggccaaggcggaaggactgcttgagtcccaggatatcaaaaccagctcagacaacatagcaagactccattccaaaaaaaaaaaaaagaaaagagaaaaagataacagtggttgcaaagggttggggagaagggaaaggagggatgaggcggaggagcacaagggagttttagggcattgaaactattctgcatgatactgtaatgggggagacatgccactgtgcatttttccaaatacacagaatgtacaacacaaagaatgaatgttaaactatgaaccttggttaataatgaatcaaaatcggttcatcagttgtaacaaatacaccacactaatgcatgatgttaacaggagaaactgcggggaagcggggtatatggaaattccctataatcaccgtgtattttttctgtaaacctgtaactactctaaaaaaataaagcccgttttaaaaagaacttgaaaaaataattcccccaaccaaaaatcaagtcaaataaaatcactgtcctcatgaaatttatgcgagaggcacagaagaaaacaaactaggcaataaatagtaaacaatagaagtaaaatatcccctaaggcatacaacaggcataataaaacaaaacaccatatgtacaactcccccatctccctcccactcctcatagatggtttaggctaaggttgataagtgggagaaaacttttggtttctgaaacatctcatagttaagcgtatctggatgggtgcggtggctcatgcctgtaatccccaacactttgggaggccaaggtgggtggatcacctgaggttgggagtttcagactggtctggccaatatggtgaaactctgtctttactaaaaatacaaaaattagctgggcctggtggtacatgcctgtaatcccaactactcaggaggctgaggcaggagatcacttgaacctgggaggcggaggttgtggtgagccgagatcatgccactgcactccagcctgggcgacagagcaagactctgcctcaaaaaaaaaagagtatccggttccccttcaaatatcaaattttcaaagacattaataactgaaatagaaagtgccagagaaatgctgaaagttgcttaatctagtaaatagctgaaataaataggaattttcttctttccaatcataagcaaaccagtcattaaaatctcagacacatacacacactcacacaaccccatctttgttcttgcttcaattttgttttagatacaatcgcaaccatacactctgtaataacatcactgccaaatactcaaaccacatgaaaaggcaaagtgccttttctattgtggaaataagcccatgttggggagaaatcctgggaatctcgcacaagaaacgcttatttacgtgtccagggccgcctctgccatgtttaccacaaataatactttaaacttcaaaagaaacgcacacacagtctaaggaacaatgaaggcttttatatagtccaatgaaaatgctttggaaaatgaaaagtataagaactgacagtacatggggatttttttgtctcagaatcttaatggagaatataaactataagcaggaaataggtcagatataacagcagacaaatcagtttcaagaagtcctacaaggaaaaatttgtgcaaaatgtcaggttgctaattattcattcagtaaacatttagggaatgtgccaagcagtcttctaagccctggggattcagcgacaaacaaaaacagatgcagcttctgctctcaaaagctgacatcctggctgggcgctgtggctcatgcctgtaatcccagcactttgggaggccgaggcaggcagatcacctgaggtcaggagttcaaaaccagcctggccaacgtggtgaaaccctatctctactaaaaatacaaaattagccaggtgtggtggcaggcgcctgtagtcccagctacttgagaggccgaggcaggagaatcacttcaacccgggaggtggagtttgcagcgagccaagatcgcaccactgcactccagcctgggcgaaaagagtgaaactctatctcaaaaaaaaaaaaaaaaggaagctgccatcctaatgggggaagacaatttgaacagattgaatagaaacacatgcagcaaaacagggtgatgggatgaaagcatgatgggctgctactttggtctaagcggtcaaggaaggcttctctgaggaggtgaggcctgaataacaagaaaccaggacggtgaagggcagagggaagaattctgggtgaaggggacaaggagccagaggcgctggagtggaaagagctgggcccacttggaggcacagggcggaggggctagcagcaggcgcagggagagctgctgagggggcagataggaggcaggcaggcagcacagggtttgtggggcagggcaaagtgcttaagaaaagccaaggaatggctttaagtagggtctgacatcatccgatttccatttttaaaagattactgtgctgtgaagcccagaaaggccgagagaaaacaaattacttagattacttcagcctacaggacttcacagggtgtctgtggggatttgggaagtgttagtgtcaaagtgtatctgagatctgaggccaggtgtggtggctcacgcctgtaatcccagcactctgggaggctgagataggcagataacttgaggtcaggagtttgagaccagcctggccaacatggtgaaaccccatctctactaaaaacagaaaaactagccaggcattgtggtgcgcacctgtaatcccagctactcaggaggctgaggcaggagaatcgcttgaaccagagattgcgccactgcactccagcctgggtgacagagcgagactccgttgtcttaatttaaaaaaaaaaaaaaaaaatctatctgaggcagaacaatagaacgcggcaaacgtgaagaaactttcagattctcctcccacctggcacgctgctcccctcccccatcgctctctcagctgatgacagggaaaggaagcccaggctgcaccccctcaccaaatgctgaccgccccaggcaggaaggaggctgcccttgctctcagccaaggccagctgcaccggtagtgcttgggattccaaggcctccatgcctgggactctcacctctgtccctgcccctactctcccttgtaccagagcaggacaggctctcacagcctacctttggaacagaacaggacacacatttaaaaaccctctctaaactctacaaacccagagaaagtcctactttcttgttccaagaaacctctttatcgttgttagttcttaacatcacatttctgctttggcctacttcaattgggcttctagatccaatattccaaggcaacggcttcttacaatatgtgcaaaacaaacagtcccttttctgttctcaaatgcattctaaaaggttggaatttcacaacttttcttctgctggttcctccacagtacacctaccaagcctttaccccaaggctttcttgaccctcttctctataaaatccaggccatcccatcctgtctcatgtcttcaaaaagatctacgtggcagagacattccaatctccatctcccccatgacatcttttcagaactccagactactcaacttctgtgctcttcagtccaaatggcgtctcaaaccagatggttatcagatggctacaggaggcattacggaatgtatcatcttctgaatgtgaaccacatcagatgcaatacagagaaacacacactctccaggcacagcaaaccatgccacagaacgctcgggctttacctgtgatgggaagcttgggctgcagtgtgtacagctgggcaggggtctgatgtgggttcattccatatctcaagggcatctgagatacgcctttgctgtactgtttcctgaaataatctgaaattgcttcatcatattgtgccgtatgagtgaatgcctacaaaaagcgtaagttttgtgagacacgagcagcactttccaaagtggggacaagatggttttctacgtccacagcaaacaacatctcctcactattcagtgctatgtggtggctgcacaacacgatgacaccatgaatgacagcaaagcactgaatggcccaacaaattacccctttccaattctttttcaatctttttttttttttttgagacaaagtctcactctgctgcccaggctagagtgcagtggtgtgatgtcagcttactgcaacttccacctcctgggttcaagcgattctcctgcctcagtctcccgagtagctgggattacaggcgtaccacaccacaccacactcggctaatttttgtattttcagtagagacagggtttcaccacattggccaggctggtctcaaactcctgacctcaggtgatccgcccgcctcggcctcccaaagtgctaggattacaggcgtgagccaccgcgcccagctctctttcaatctttttgacagaacacctctgtaatagctggtttctccatttttaacaaaaggatagcagctccaagcaaagctttggtaggtaatgcaatctaccaagaactgaatactgcttggtctggcaaacactgcactccatggatgtaacttacaatatcatgaaagtgcatcccaccttcaaggctaactggcgtctagtctccaaggaggtgtccttactctcggagctctgcatctccgtggacaccaccacatagtcctctggttcacacaccactgtcactcgagcgtggtttttggctgcagctctcagtaaggttactccacctttgaagaacaaaagacttattgatttttgtatgtccctatcagcttatgaaccgcttttaaataaagtcattgtgcatgtacttccattttaattcctgacttaaaaatcaggaaaataagctgggcgcagtggctcatgcctggaatcccagcactttgggaggctgaggcaggtggatcacctgaggtcaggagttcgagaccactctggccaacatggcgaaattccatctctataaaaaatacaaaaaaaattagccaggcgtggtggtgtgcacctgtaatcccagctactcaggaggctgaggcaagagaatcacttgagcccaggaggcagaggttgcagtcagccaaaactgtgccactgtactccagcctgggtgacacagcaagacttcatctcaaaaaaataaaaataaaataaataaataaataaatcaggaaaataacccagatgcatctctgcatacaaaaaaagcttagaaaatataaatgaaaaaaagaaaatataaatgaattatagtaatcccaaaacacaatccagaagtagctattttctttctatttcctcagtaataaaattcaaaatgaaatctcactaaaatttaaataatcctctcctctcagtcttctaaaatggtttttctgacttaccaatgtcaatttgctccacagcctcctcaacagttacacctggagaagccactgtctttacaaagggatagagattgcaggcaacaactctataaaacaaagacgaatctggctgtcattaaaaagtccactcatgtggtttacagctatctaggaaaaaaacaagtcagtactattaacgtaaacaaaagccatagagaactaatatataaactgtgagcctgacattttagaattctaacgagtgtgttaaaaagtcagggtgtcctgctggggtcgagcagaggtaatttagtcagcaggtgctccccggatctgaatacacagtactccctccacgtcacactgcaacgctgctgcttgcataaccttagctacccagaaccaccaagccatgaaaaatttatagaacattttaatagaagtataaagtacaagattacagataacattctcattttggaatgtgatcactattatccagaacattagaatttagattctttttccagtggattggcacaatgatgaaaaatatcagagcattctaagggtgaaacatttgttttgccttcagagaggtcataaaaatgatggtaacaggaatgccaaaagaataatttaaaatacagaagtaatctaaaattaacaatcagtgaagtacagttccactcctaggcctgtagtgcgaggagctctacgggcaggctccaaacaagcctaactgctgaaaactaattaaaaaaaacaaaaacaaaaacagaagaacagaattggaggggaaacattttgcagctcatctttattaagtctactgccctcctactaaaaccagacaaatgtcacaagaaaactagagaccagtatctcttaggtatcttcacacaaaaactctcaacaaaacactaacaaactgaatccagaaaaatgcaaaaaccacctgatcatctcaatagatgtacaaaaaggatctgacacaattcaacaggtattcatatagaaacactcagcaaattaaaaatagaagggaacttactgaacatgacaaagtgtaagaaaaacccacagctaacatcatacttgatggtgaaagactaaaatacttgtctactcaactcagaaacaagacaagaggccaggcgcagtggctcatgcctataatcccagcactttgggaggctgagccaggcagatcacctgaggtcagaagttcgagaccagcgtggccaatatggtaaaaccccatctctactaaaaatacaaaaattagccgggcaaggtggcgcacgcctgcaatcccagcactttgggaggctcaggaggatggatcaactgaggttggaagttcaagaccagcctgaccaacatggagaaaccccatctctactaaaaatacaaaattagccaggcaaggtggcacatgcctgtaaccccaggctgaagcaagataatcacttaaacccgggaggcggaggtcacagtgagccaagatcacgcaactgcactccgccttgggcaacaagagcaaaacgccatcttaaaaaaaagaaacaaaacaataatgtccattcttaccacttctactgaacactgtactagaagttctagccagagcaattagcaaagaaaagaaaataaaaggcatcaggactggaaagaaagcagtgaagttatatctatttgcagatgataccacctagtatacagaaaatcccaaattcacaaaaataaccttttagcactaataaatgagttcagcaaagccgcaggatatatgatcaatatataaaaatcaattgtattaaaatatacattagcagtgaatgagaaaaaaattaagaaaacaattctacttcacaagacaatgttaaagaaattaaagacaccccatgttcacagactggaagatttaatattcttaggatggtaacatttcccaaattgatctacagattcaacacaatacctatgaaaattccaactgcgttgttttgcagaaatggacaagctgattctaaaaatcatgtggaaatgcaagggactctgaagagtcaaagtcatcttgaataaaaaaaacgaagttggggccgggcgcagtggctcacgcctgtaatcctagcactttgggaggccgaggcgggcggatcacgaggtcaggagatcgagaccatcctggcgaacacggtgaaaccctgtctctactaaaaatacaaaaaaattagctgcgcatggtagcgggcgcctatagtcccagctactcgggaggctgaggcaggagaagggcgtgaacccgggaggcagagcttgcagtgagccaagatcgcgccactgcactccagcctgggcaacagagccagactccgtctcaaaaacaaaacaaaacaaaacaaacaaacaaacaaaaaacagaaaaatgaagttggaggactcacacttgctgatttcaaaatttgttaccagcctatagtaatagtgtagtactggcataacagcaggcacatggaccaagggtgccaagaccattcgattgggcaaagaacagtcttttcaacaaatgatgctgagacaactagacagccacatacaaaataaaataagacttctatttcatgtctgtatatgaaaattaacttaaaatgaaccaaagacttaagtataagagctaacactaaaataactcttaaaagaaatgtacacacgccgggtgcggtggctcacacttgtaatcccagcactttgggagaccgaggcaggtggatcacctgaggtcaggagttcaagaccagcctggccaacatggagaaaccccgtctctactaaaaacacaaaaaaattagctgggcgtggtggcgggcacctgtaatcacagctacttgcgaggctgaggcatgagaatcgcttgaacccaggaggcgaaggttgcagtgagccgagatcacgccactgcactccagcctgggcaacaagagcgaaactccatctcaaaaaaaaaaaaaaaaagaaagaaaatatacacaaaaatcttcatcactgaggattaggcaatggtttcttagacatgacaccattaacacaagcaacaaaagaaaaaataaattcaacatcatcaaaattaaaaacttttgtgggccgggcatggtggctcaagcctgtaatcccagcactttgggaggccaaggcgggcagataacccgaggtcaggaattcaagaccaacctggccaacatagtgaaaccctatctctattaaaaatccaaaaaaattagccaagtgtggtggcgggcacctgtaaccccagctactcatgagactgaggcagaatcgcttgaatccgggaggtggaggttgcagtgagccaagatgacgccactgtgctccagcctgggcgagagtaagactccgtctcaaaaaaaagaaaaaacttgtgatgatcaaaggaaaccatcaagaaaggctaacacaatccatagaatgagaggaaataaacttacaactcaataataaacagataaatacctcaactaaaaatggacaagaggtctcaacagacatttctccaaaggaggtaaacaactggctaggaagcacatccaacggtgttccacattatttggaaagcaaattataaccacaatgagatgccacttcagaacccctagaatgactagaatcaaaaagacagatagtaacgttgatgaggatgtggagaaactggagccccatccagcacactgctgctggaaatgtaaaatggtgcagcactctgaaatacagggtcgcagttcctcaaaagctaaacatagttaccatatgaccaaggaattccactcctaggtatataccccaatgaaacgaatgtgtatgtccacacaaaaacttatacacaaatatttatagctgcattattcataatattcaaaaagtagaaacaacccaaatgtccatcaacaaatgaactgataaacaacagggtatgaccacacaatggattatttttcagcactaaaaacgaaatgaactattgatactatgctataacatggataaaccttgaaaacatgctaagtgaaagaagccaggaacaaaaaaaccatatattgttagattctatttatatgaaaatgtccactacaggcaaatcagacacgtaacaattggtggttgccgaggctggaggacgttggggaaatggagaataaatgcgtatgggtatagggtttagggtgggggtgatgaatatgttttaatattggatgtggcaattaatatggtttggatgtgtgtcctctccaaatctcattttgcaatgtgatccccaatgttggaggtggggcctggtgggagatgattgggtcacgggggagatcccttatgaatggcttggtgccctccccataatgagtcctcactctgagttcatgtagagctggttgtttaacagagccgggcaggccgggcgtggtggctcacacctaatcccagcactttgggaggccggggtgggtgcatcatttgaggtcaggagttccaagagcagcctgaccaacatggtgaaaccccatctctactaaaaatagtaaaaatgagttgggcgtggtgacaaacgcctgtaatctcagctactcaggaggctgaggcaggagaatcacttgaacacaggaggcagaggttgcagtgagccaaggtcatgccactgaactccaacctgagcaacagagcgagaccctgtctcaaaataaaattaaattaaattaagaataaaagttaaaaagtaaaagagctgggcaactcctcctctccctctctcttgcttcttctctcatgggatatgccagctccccctctgctttccactgtgattataaacttcctgaggccctcaccagaagttgatgctggcaccacacttcttgtactgtctatagaatcacgagccaaaataaacctgttttcataaattacccagcttctagtattcctttatagcaatgcagaacagactaacacagtaatggttgcacaacttctgtgaaccactgatttgtacattttaaattggtgagttgtatggtatgtggattagatctcaataaagctgttacgtatatttaagagaaacagccagtatctttatgcaaataactcagttagttgtgcaagttgataattcctaatatgaaaacctgaaatctgaaatgctccaaaatctgaaactttttgagcgtcaacatgacacttggaggaaatgctcactgaagcatttcggatttctgagggacgctcaactggcatgttacctgcaaatattccaaaatctgaagaaatctgaaatccaagacacttctgggtcccaagcatttcagataagggatactcaacctatacatactaagatttagattgtaacttcctaaaaaacttcagtatgtattttggtatataaatctggccaaaccaaaatgcttacacacatacaaagtcacacagaaggtacgcagtaaaggtttatagtgagtaagaaaacacagggacaccaggtttggcatagtctttgtcgttggttcgtaatgtgttaaaagtttaatttcaggtttttaccttataagattgaaatcaagtctggccatgtcagcattatcttctggaatattacgagctaggattcctaatttaaaaaagttaaacaaagtcattagcttcaaaaaacaagtcagattaaggaatgtctttcaacatagtgtcttcaatcaaattaaatcccattgattaaaaaaaaaatttatttttttgagatggtctcactctatcgcccaggctggattgtagtggcgtaatcacaactcactatagccctgacctccagggctcaagtgatcctcctacctcagcctcccaagtagctgggactacaggaatgcatcaccacacccagctggtcttttcaattttttaaagagaaggggggtgttgccatcttgcccagggtgatcttaaactcctgggctcaagcggttctcctgtctcagctctccaagtgctgggactacaggcaagagccaccatgcccagcataatattcttaattaaagaatatcaaacatgtagttggggagttatcaaacaaaaaaattaggattaacagatatatctggctggccacagtggctcatgtctgtaataccagcactttgggaggctaaggtgggggatcgcttgagcccaggtgttcgagaccagcctgggcaacaaggtgagaccccgtctctataaaaaatacaaaaattagctgggtgtggtggcacatgcctatagtcccagctacttgggaggctgaggtgggagaactgcttgagcctgggaagcagacagaggctgcagcgagccatgatcgcaccactgcactccagcctgggcaagagtataatcctgtctcaaaaaaaaaaaagagagatatatttaaaaatctttttcttcagttcttgatcccagtttcttgagcaataaaatgtaaaaaaggtaaaaggaattttacaaaactgttgctagttccagaacagtctatgcgtttctgtaagtcaatggcttgtctgatgatctgtagccattcagctttattcaccaaattgtaccacaactgacaaactaggaggataccagtttcttgccgtttcggtttatatacaattcattcttaggtgaacgaacaaatgtaaagcactttgaaaaaaaaaattcctttgcaagtcagttacgctgtgatgctttcttctaagtttgaactattttaaaagtattttcatacgatgaaatctaaatatcttcagcaacttgagcaggaaacaccagactctaaattaggtatcaggtcaggactaatagcaagcagaggaaaacaatctgaattcatcaagtagtaagacaagctggattttaaaccagttctaacgtattatgaaatctggcagcaacttcataacattaactttatatgtaaatttctcttgagcctttttcttcctttattttaggacgctatcaactaaaatattcctggaaaccactgactgtttttacattaaagataccaaccacttaccagcatggactgcaggatgcaaagttttcacacgtccccccaacatttcaggaaatcccgtcaactcagagacatctctgaaaaacagataaacagaatggtactactggtgtggctgaattgtaattttcactattcaaagcatctactgttttcatttcaagagattttgagcctgaattcaacgtgaaaattctgattttttaaagtatttcactatattttatttcttaagtaaaattctatttacttccgaaaaaaagtttatttaatttatttcctaactgttgtgtatgttccgtacgattaagggcgaattccagcacactggcggccgttactagtggatccaacagtctccacgtaaacggtcaaagtccccgcggccctagacaaatattacgcgctatgagtaacacaaaattattcagatttcacttcctcttattcagttttcccgcgaaaatggccaaatcttactcggttacgcccaaatttactacaacatccgcctaaaaccgcgcgaaaattgtcacttcctgtgtacaccggcgcacaccaaaaacgtcacttttgccacatccgtcgcttacatgtgttccgccacacttgcaacatcacacttccgccacactactacgtcacccgccccgttcccacgccccgcgccacgtcacaaactccaccccctcattatcatattggcttcaatccaaaataaggtatattattgatgatgattgtttaaacatgatgagctcagc

SEQ ID NO: 3 - DNA, 35067 bp, Homo sapiens
tcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattacgggcccatatagtttaaacatacatcatcaataatataccttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatggctgccgctatgcccggggggaggcctaatcggccttagcgatcgcttagggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggcagccgccaccatgaattgtcctgtactgagcctcggtagcggctttttgttccaagtcatagaaatgctcatctttgcgtacttcgcgtcaatatctcttacagagtctcggggtctcttcccaaggcttgaaaatgtaggagcgtttaaaaaggtttccatcgtaccaacccaggctgtatgtggccttcctgaccgcagtactttttgccactcttccgccgctgctgagtcaattcagttttgcactcagcggttctgcattcaagattgcccctatcggtcttctcacccaacatatacggcactgttttctgcggggcttagttcctgtattacccctgacaagaatgatcttcaccctaacgcgcactcaaatagtgcatcatttatcttcggaaaccacaaatcctgtttttcttctcctccgtcccccaaactgatggcctcctttacgttggcggtttggcttaaaccggaacaacaaggagtaatgtgcgtgattgagaagacggttgatggacaaatcgtctttaagctgacgatatcagagaaagagacgatgttttattaccggacggtgaatgggctgcaacccccgattaaagtaatgactctcggtcggatcctcgtgaagaaatggattcacctgagtgtacaagtccatcaaactaagatttcattttttatcaacggagtcgagaaggaccataccccatttaacgctaggacattgagtgggagcattacagatttcgcctccggaacggtacagatcggccaatccttgaatggattggaacaattcgtcggaagaatgcaggacttccggctgtaccaggtagcactgacaaatcgagagatccttgaagtctttagtggggacctgttgcggctgcatgcccaatcacactgccggtgtccaggttctcacccgagggtacacccgctggctcagcggtactgtataccaaatgatgccggggacacagcagataacagagtcagtcgacttaacccggaggcacaccctctctcattcgtaaacgacaacgacgtggggacttcttgggtgtctaacgtcttcacaaatataacacaactgaatcaaggcgtcaccatctccgttgacctcgagaacgggcagtatcaggtgttctatattattatacaatttttcagtccacagcccacggagatacgaatccagcggaagaaagagaattccctggactgggaagactggcagtacttcgctcgcaattgcggggcgtttggcatgaagaataacggagacctggagaagcctgattccgtgaactgccttcagctctcaaacttcaccccctactcaagagggaatgtgacattctcaattcttactccgggccccaattatcggcccgggtacaacaacttttataacacgccttccctgcaagaatttgtaaaggcaacccaaatcagatttcacttccatggacaatactataccactgaaacagccgttaatctgaggcaccgctactacgccgtggatgaaatcacgatcagtggacggtgtcagtgccatggccacgctgataactgcgacacaactagccaaccatacagatgcctctgcagccaagaaagtttcactgagggccttcactgtgatagatgcctgcccctttataacgataagcctttcaggcaaggagaccaagtttatgcgtttaactgcaaaccatgccaatgcaacagccattccaagtcctgtcattacaacatttccgtcgacccttttccatttgagcattttaggggagggggcggagtatgtgatgactgcgaacataacactactggtagaaactgtgagttgtgcaaagattacttctttcgccaagtgggtgcggatccatccgcgatcgacgtatgtaagccctgtgactgtgacaccgtcgggacgcggaatggatctatcctttgtgatcaaataggtgggcaatgcaattgtaaacgacacgtatcaggtaggcagtgcaaccagtgtcaaaatggattctacaatctccaggaactcgaccctgacggatgtagtccatgcaactgtaacacctctggcaccgtagacggtgacataacatgtcatcagaatagtggacagtgtaaatgtaaggctaatgttatcgggctcaggtgcgatcattgcaatttcggttttaagttcctccgctcatttaatgatgtagggtgtgaaccgtgtcagtgtaaccttcacggtagtgttaacaagttttgtaacccccattctggtcagtgcgagtgcaagaaggaggccaaggggttgcaatgcgatacttgccgcgaaaatttttatgggcttgatgtaaccaactgtaaggcgtgcgactgcgatactgcaggatctctgccaggaactgtgtgcaacgcgaaaactggacagtgcatttgtaaaccgaatgtggaaggacgacagtgcaacaagtgcctggagggaaatttttatctccggcagaacaactcatttctctgtctcccatgtaattgcgataaaactggcaccattaatggcagcttgctttgtaataaaagcactggacaatgcccctgcaagctgggagttacggggctgcggtgtaaccaatgcgaaccacaccggtacaatctcaccatcgacaatttccaacattgtcagatgtgtgaatgcgattccctcgggacactccctggcacgatatgtgaccccattagtggccaatgcctgtgtgtacccaaccgacagggtcgacgatgtaatcagtgtcagccaggattttacataagcccgggaaacgccacaggttgcctgccctgctcatgtcacacgacgggagcagtcaatcacatatgcaatagcctcactgggcaatgcgtgtgccaagacgcaagcattgctggtcagcgctgtgatcagtgcaaagatcactacttcgggtttgacccacagacaggtcggtgccagccgtgcaactgccaccttagcggtgcattgaacgaaacatgtcatttggtaacgggtcagtgcttttgcaaacaatttgtaacgggttccaagtgtgacgcatgtgttccttctgccagccatcttgacgttaacaatctccttggttgttctaagactccatttcagcaaccaccgccaagaggccaggtccaaagctcatcagccatcaacctctcatggtcacctcccgattcaccgaacgcgcactggttgacctactcattgctccgcgatggattcgagatttataccaccgaggaccaatatccgtattccattcagtactttctggacactgacttgctgccctatactaaatatagttattatatcgagacgaccaatgtgcacggttctacgcgatctgtggccgtcacatataagactaagcctggagtgcctgaaggtaatctgactttgagctacataatcccaattggatccgacagcgtgacccttacgtggaccacgctttccaaccaatcaggaccaattgaaaaatacatcttgtcttgcgctccgttggcggggggtcaaccctgcgtctcatatgagggccatgagacaagtgcgactatatggaatctcgttccctttgcaaagtatgacttttctgtacaagcctgcacatctggcggatgcctgcatagtcttccaatcacagttaccacagcacaagcacccccccagaggttgagcccgccaaagatgcaaaagatttcttctacggaactccatgtggagtggagtcctccggcagaactcaatggtattatcattaggtatgaactgtacatgcgacggcttagatctaccaaagagactacgagtgaggagtcacgcgtgtttcagtcatctggctggttgtcaccccattcattcgtggagtctgcaaatgaaaacgcgttgaagcctcctcaaacaatgactacgataactggacttgagccatatactaaatatgagtttagagtactggctgtcaacatggccggctccgtctcaagtgcatgggtttccgagcgcacaggcgaatccgcgcctgtcttcatgataccccccagtgtttttcctctgtctagttactccctcaacatatcttgggaaaagcctgcggacaacgtgaccagaggtaaggtcgtgggctacgatatcaacatgctgtcagagcagtccccacagcagagcatcccgatggccttttcacaactcttgcatacggccaaatcacaagaactgtcatacacggtcgagggacttaaaccttatcggatctacgagtttacgatcacgctgtgcaactcagtcgggtgtgtcacttctgcgagcggtgcaggccagacgcttgcagcagcgccagcgcagctcaggccccctttggtcaagggaataaattctactacgatccatctccggtggtttcctccggaggaactgaatggccctagtcccatttatcagctcgaaaggagagagtcttcactgcctgcactcatgaccactatgatgaaaggaatccgctttattggaaacgggtactgcaaattccccagctccacgcaccctgtgaatacggattttacagggatcaaggcctcttttagaacgaaagtgccggagggcctcatcgttttcgctgctagccccggcaaccaagaggagtatttcgcccttcagttgaagaagggaaggctgtacttcctgttcgatcctcagggaagtccagttgaggttacgacaacaaatgaccatggtaaacaatacagcgatggtaaatggcacgagataatcgcgattagacaccaggcgtttgggcagatcacactcgacggcatttacactggatccagtgcaattctcaacggtagcacagttattggcgacaatacgggggtcttcttgggcggtctgccacgatcatacactatacttcgcaaggatcctgaaataatacagaagggttttgtcggatgccttaaggacgtgcattttatgaaaaactacaatccttccgctatatgggagccactggactggcagtcttcagaggaacaaatcaacgtgtataattcttgggaagggtgtcccgcgagccttaacgaaggggcacaatttttgggggcaggtttcctcgagttgcacccatacatgtttcatggggggatgaatttcgaaatttctttcaagtttcggacggaccaattgaacggcttgcttctgtttgtctacaacaaggacggccctgatttcctggccatggagctcaaaagtgggatactgaccttccgccttaatactagtctggcgtttacccaagtagatctgctgcttggcctttcttactgcaacgggaaatggaacaaagtgatcatcaaaaaagaaggctcctttatttctgcatctgtgaatggcctgatgaagcatgcctccgaaagtggtgaccaaccccttgtagtcaacagcccggtatatgttggggggatcccacaagaactcttgaacagttaccagcacttgtgcctcgaacaaggtttcggcggatgcatgaaggacgtgaagtttacccggggggccgtagtcaaccttgccagcgtatcatccggggccgtgcgggtcaatttggacggatgcctgtctacagattccgcagttaattgtcgagggaatgattcaatacttgtatatcaagggaaagaacaatcagtatacgaagggggcctccaaccttttacagagtatttgtatagagtcattgcaagtcatgaagggggatccgtttactcagactggtcacggggccgcacaaccggcgccgcgccacagtccgtccccacaccttccagggtgcggtcactgaacggttacagcatcgaagtgacgtgggatgaaccagtagtaaggggtgtgattgaaaagtatatattgaaagcttacagtgaggattccacacgccccccaaggatgccatccgcgtctgcggaattcgttaatacttccaaccttacaggcattttgacaggactgctcccttttaaaaattacgctgtcacacttactgcttgtaccctcgcggggtgcacagaaagtagtcatgcgctgaatatctccaccccgcaggaagcccctcaagaggttcagccgccggtcgcaaagagtcttccgagtagtctgctcctttcctggaacccccctaaaaaagcaaacggtatcatcacacaatactgcctctacatggacgggcggttgatttactctggtagtgaggaaaactacatagtaaccgatcttgctgtctttactccacatcaattcctcctttctgcgtgtactcatgtaggctgcacgaactcaagttgggtacttctctatactgcccagttgccacctgagcacgttgactctcccgtattgactgtattggacagcaggaccattcacattcaatggaagcagcctcggaaaataagcggcatccttgaaaggtatgttctctatatgtctaatcacacccatgacttcacaatttggagcgtcatttataatagtacagaactgtttcaggatcatatgttgcaatatgtactccctgggaataaatacttgataaagctgggtgcgtgcaccggaggtggttgtacggtgagcgaggcatctgaggcgcttacggacgaggacatcccagagggtgtacccgccccgaaggcccactcttacagccctgacagcttcaatgtgagctggactgaacccgaatacccaaatggcgtaataaccagttatggactgtacctcgatggtatattgatacataattcttccgaacttagttatagggcctacggatttgctccgtggagcttgcacagtttccgggtccaggcttgcaccgcgaaaggttgcgccctgggtccgctcgtggaaaatcgaacactcgaggccccacctgaggggaccgtgaatgtgtttgttaagacacagggaagtcgaaaagcgcatgtccgatgggaggccccatttagacctaatggcttgcttacgcactctgtgttgttcacggggatcttctacgtcgaccctgtaggtaataattacacattgctgaacgtcaccaaggtgatgtactccggagaggaaactaacctttgggtactgattgacggcctggtaccgtttacgaactatacagttcaagtgaacatcagtaacagccaagggtccctcattacggatccgattactatcgcgatgccgccgggtgctcctgatggagttctccccccccgcctttcatctgctactcctacttcattgcaggttgtgtggtcaacccccgcgagaaacaacgcgcctggtagccccagatatcaacttcaaatgcggagcggggattccacccacggtttcctcgagctcttttccaatccttcagcaagcttgtcctacgaagtttctgatctccagccatatactgaatacatgttccgactggtggcgagcaatggatttgggtcagcccacagttcttggatcccctttatgaccgctgaggacaagccagggcccgtggttccgcccatcctgctggatgtcaaaagtcgaatgatgctggtcacgtggcaacatccgagaaagtccaacggcgttataactcactacaacatttacctgcatggtcggctttacttgcgcaccccaggaaatgtaacgaactgtaccgttatgcacctccacccttataccgcctacaagttccaggtcgaggcatgtacgtccaaaggatgctcacttagccccgaaagccagaccgtttggacgcttcctggggctccggaagggatccctagtccggagctctttagcgatacacccaccagtgtgataatcagctggcagccccctactcatccgaatggtctcgtcgaaaatttcacgatagaaaggcgagttaaaggtaaagaggaagtgaccaccttggttactctgccccggagccatagtatgcgctttatagacaagacctccgcccttagcccctggaccaaatatgaataccgggttcttatgtccacgttgcatggagggacaaattccagcgcttgggtagaagtcactactcgaccaagccggcccgcgggcgttcaaccacctgttgttacggtactggagcctgatgctgtccaagttacgtggaaacccccactgatccagaatggtgacatactgagttacgagatacatatgccagaccctcacataacattgacgaacgttacctctgcagtgctttcccagaaagttactcacctcattccgtttacgaactatagtgtcacaatcgtggcctgtagcggtggtaacggatatcttggagggtgcaccgaaagcctccctacttacgtgacgacgcacccgacagttccgcagaatgtaggtcccctgtccgttattccgttgtccgaatcctatgtcgtgatatcttggcagcccccctctaaacctaatggacccaatctgaggtacgagctgcttcgacgcaagatccaacagccattggcgtccaacccgccggaagacctgaatcggtggcacaatatttatagtggcacgcaatggttgtacgaagacaagggcttgtccaggttcacgacctacgagtatatgcttttcgttcataattccgtcggatttaccccgtctcgcgaagtgaccgtcaccaccttggctggcttgccagaacgcggggcgaacctcactgcttcagtcctgaaccacaccgcgatagatgttcggtgggctaagccaactgtgcaagatctccagggcgaggttgaatactataccttgttttggtcctctgcaacgtccaatgatagtctcaagatattgccggatgttaacagtcacgtaattggacatctcaaacctaacacagaatactggatatttatttccgtgtttaatggagtgcactccataaacagtgctggactgcatgcgacgacttgtgatggtgagccacaggggatgcttccccctgaagtagttatcattaactcaaccgcagtcagagtaatttggacttctccaagcaacccaaatggagtagtaaccgagtactctatttatgtgaacaataaactgtacaagaccggcatgaacgtacccggctcatttattttgagagacctgtccccgtttacgatttacgatatacaggtggaggtctgtacaatatatgcatgcgtcaagtccaacggaacacaaatcactaccgtcgaagatacaccctcagatattcctacgccaaccattaggggtattacgtctaggagtcttcaaatagactgggtgtcaccccggaagcccaacgggattattctcggatatgacctgttgtggaagacttggtatccgtgcgccaaaacacagaaattggtccaagatcagtcagatgaattgtgtaaggcggtgcggtgtcaaaaacccgagagtatctgcggacacatctgttatagctcagaggcaaaagtatgctgtaacggtgttctgtacaatcccaaaccaggccatcgctgttgtgaggagaaatacatcccgttcgtgctgaactcaacgggggtctgttgcggtggtcgcattcaagaagcccagcccaaccaccagtgttgctctggttactacgcccgcatcctcccaggggaggtatgttgccctgacgaacaacacaacagggtaagcgtaggcattggcgactcctgttgtggccggatgccgtatagtactagcggtaaccaaatttgctgcgcgggaagacttcatgatggacacggacagaaatgttgcggccgccaaattgtatcaaatgacctcgaatgctgcggcggcgaagaaggcgtagtttacaataggctgccggggatgttctgttgcggacaagactacgtaaatatgagcgatactatttgctgttctgcaagctctggggagtcaaaggcccacataaaaaagaatgacccagtaccagttaagtgctgcgaaactgagctcattcccaagtcacagaaatgttgcaatggggtaggatataatccgttgaagtacgtctgctcagataagatttcaaccggtatgatgatgaaagagaccaaggaatgtagaattctgtgccccgccagtatggaagcaacggaacactgtggtcggtgcgactttaatttcacgagtcacatctgcactgtcatccgcggctcccataactcaactggcaaggcctctatagaggaaatgtgcagttccgcggaggaaaccatacatacgggaagcgtcaatacgtattcttatactgacgtcaacctgaagccctacatgacgtacgagtaccgaatcagcgcgtggaattcatacggccgcggattgagtaaagcagtcagggctagaacgaaggaggatgttccgcaaggcgtaagcccacccacttggacgaaaatagacaaccttgaggatacaattgtccttaattggaggaagccgatccagagtaacggtcctattatttactacattcttctgcggaatgggatagagaggtttcgaggcactagtctgagtttttccgataaggaaggtattcagccatttcaagaatacagctaccagctgaaagcatgcacagtggcgggatgtgccacctcaagcaaagtagtagccgcgactacccaaggcgttccggagagtatacttccgcccagtatcaccgcgctgagcgccgtagcattgcacctcagctggtcagttccggaaaaaagtaatggcgtcattaaggaatatcaaatacggcaggtgggaaaaggccttattcacactgatacgaccgaccgacggcaacatacagtgactggtctccaaccgtatacgaattattcattcacactgaccgcctgcacaagtgctggatgtacctctagtgagcccttccttgggcaaactctgcaagcggccccagaaggcgtttgggtcaccccacgccatattattatcaactctaccacggttgagctctactggtcccttccggagaaacccaacggcctggtatcccaatatcaactctccagaaatggtaacctgctctttttgggcggtagtgaggaacaaaatttcacggataagaatctcgagcctaactcaaggtatacttataaacttgaggtgaaaaccggaggcggcagttctgccagcgacgattatatagtacaaacacctatgtccacaccggaggaaatctatccaccgtataacataacagtgataggtccttactcaatatttgtagcgtggatcccgccagggatattgatacccgagattcctgtagagtataacgtgttgctcaatgatggctccgtgactccgcttgcattctcagttggacatcatcagagtacgctccttgaaaaccttacgccatttactcaatatgaaatccggatccaggcgtgccagaacggtagctgtggtgtgtcctctcggatgtttgtcaaaactcctgaagcggcaccaatggacctcaacagtcccgtgcttaaggctttgggctcagcttgcatagaaatcaagtggatgccacctgagaaaccaaatggtatcataattaactatttcatttaccggcgccccgcagggatcgaggaggaaagcgtgctttttgtatggtctgaaggggcactggaattcatggacgaaggagataccttgaggccatttaccctttacgagtaccgcgtgagagcttgcaactctaaggggagcgtcgaaagcttgtggtctctcacgcagacccttgaggctcctccccaagatttcccagcgccgtgggcccaggcgacatctgcacacagcgtgttgcttaactggactaaacctgaaagcccgaacggcatcatctcacattatagggttgtttatcaagaacggccggacgatccgactttcaattctcccacggtgcacgcctttacagtaaagggtactagccatcaggcccacctctacggtctcgaaccattcacgacgtatcgcatcggcgttgtagcggccaatcatgctggtgagatacttagtccgtggacgctgatacaaactctggagtctagcccctccggtttgcgcaacttcattgtggagcaaaaagagaatggtcgagctcttctccttcaatggtccgaacccatgcgcacgaatggcgtgataaagacttataatatattctcagatggcttccttgaatactcaggtttgaaccgacagttcctttttaggaggcttgacccattcactctctacacgctgacgctcgaagcgtgtaccagggccggttgtgcacattctgcaccacagcccctttggacggatgaggctccccctgattctcaactcgccccaacggtgcatagcgtcaagtcaacctcagtagagcttagttggtctgagccagtgaaccctaatggaaaaattataagatacgaagtgatacgccgatgtttcgaaggtaaagcttggggcaatcaaacaatccaggccgatgagaaaattgtatttacagaatataacaccgagaggaacacgttcatgtataacgataccgggctgcaaccttggactcaatgtgaatataaaatttatacttggaattcagcgggccatacctgctcctcatggaacgtggtgcggactctccaagcaccgccggagggcctttcaccgcctgtaatctcttacgtttccatgaatcctcagaagctgcttatctcctggataccacccgaacaaagcaacggaatcatacagtcttatcggttgcagaggaacgagatgttgtatcctttttccttcgaccccgtcacttttaattatacagacgaggaactgttgccatttagcacctattcctacgcccttcaagcgtgtacgagcggtggctgctccacatccaaaccgaccagcattaccacacttgaagccgctccaagcgaggtctctccacctgacctctgggccgtttctgctacgcaaatgaatgtgtgctggtccccgcccacggttcagaatgggaaaatcactaaatacctcgtaaggtatgacaataaagaaagtctggctggacaagggctctgccttctggtcagtcacctgcagccttatagtcaatataattttagtctcgtagcgtgtaccaacggcggctgtacagcctctgtttcaaaaagcgcttggacaatggaggccttgcccgagaacatggactctccgaccctgcaggtgaccggatcagaatccatagaaatcacgtggaagccaccacgcaatccgaacggacagattcgaagctacgaattgcgccgggatgggactatcgtgtacacagggctggagactcgatacagggacttcaccctgactccgggagtggaatattcatatactgtcacggccagtaactcacagggtggaatcctgagtcctctggtgaaagatcgcactagtcccagtgcgccctctggcatggagccaccgaaattgcaagcaagagggccccaggagatcctggtcaattgggatcctcccgttaggactaacggtgacattattaactacaccttgtttatcagggagttgttcgaaagggaaaccaagattatccacattaatactacccataacagcttcggcatgcagtcatatattgtaaaccaattgaaaccgttccaccgatacgagattaggatccaggcatgcacgaccttggggtgtgcatccagcgactggacgtttattcaaacgccagaaattgcgcctttgatgcagccgcccccgcacctggaggtacagatggctccgggcgggttccaacccacagtctctcttctctggaccggacctctgcagcctaatggaaaggtcctgtattatgaactttacagacgacagatcgccactcaaccccgcaaatccaatcctgtgcttatctacaacgggtctagcacgagcttcattgatagcgaattgctgccttttaccgagtacgagtaccaagtgtgggcagtgaactcagcggggaaagcgccttcctcttggacgtggtgccggacgggacctgcgccacctgaggggctgcgggcacccacgttccatgttatcagctcaacgcaagctgtcgtaaatatctccgctccaggtaaaccgaacggtattgtatccctctataggttgttctcttcctcagcacacggcgctgaaacagtactgagcgaaggaatggcaacccagcagaccctgcacgggttgcaggcctttacgaactatagcattggagtcgaagcgtgcacctgcttcaactgttgtagcaaaggccccaccgcggaactccggacccatcctgctccaccctctggcctgtcctccccacaaatagggaccttggcctctcgcacagcgagtttccgctggagtccgccgatgtttccaaatggggtcatacattcatacgagcttcagttccatgtcgcgtgcccccctgactctgcgctgccttgcaccccaagccaaatcgagactaagtacacagggctgggccagaaagccagtcttgggggtctgcaaccctatacgacgtataagcttagggtagtcgcccacaacgaggtaggtagtacggccagtgaatggataagtttcacgacacaaaaagagttgccacagtaccgggccccgttctccgtggacagcaacctcagtgttgtatgtgtcaattggtctgatacattccttctgaatgggcaacttaaggaatatgttttgacagacggggggagacgcgtttactctgggcttgacacgactctgtatattccacggaccgcggataagacttttttttttcaggtgatttgcactactgacgaggggtctgttaaaaccccgttgatacagtacgatacctcaacgggattgggtctcgttcttacaaccccggggaagaaaaagggcagcaggagcaaatctacagagttctatagtgagctgtggtttattgtcctgatggccatgttgggtctcattcttttggcgatatttctgtcactcatattgcaaagaaagatacataaggagccttatatcagagagcgccctcctttggttcctttgcaaaagagaatgtcaccgcttaacgtttatccgcctggtgagaatcacatgggactggctgataccaagatccctaggtctggtacacctgtgtctatccgcagtaaccgctccgcttgtgtactgcgaataccgtcacagaatcagacaagtctgacatacagccaaggcagcctccaccggagtgtatctcaactcatggacattcaggataagaaggtcctcatggacaacagcttgtgggaagcgatcatgggtcacaactctggcctctatgtagacgaggaagacctcatgaatgcaataaaggatttttcaagcgtgactaaagaacggacgactttcacggacacgcacttgtaataaggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactggccggccatcgatgtaagaaattatattacaagctaaagcaacagctgtgggtggaagaagagaagaaataatgtgttttttctttcttctttgatggcaaactcaacagactctgcctaaagcagaactgtagttaaaaggagactgcaacttcagagttttccattacctagtttctcaacactggcagatttttttaggagctagtatctcttgtgaataaacataagaagttcagcacacagaaagaaatggtgggccagcactaattaaaaacaaaaatgagccgggtacaatggctcacccccatgaccccagcactttgggaggccgaggtgggtggatcacaaggtcaggagttcgagatcagcctggtcaacatggtgaaacccccgtctctactaaaaacacaaaaattagctgggtgtggtggcgggcgcatgtaatcccagctactagggaggctgaggcaggagaatcgcttgaacctggaaggtggaggttgtagtgagccgaaaccgcgccactgcactccagcctggccaacaagagcgagactccgtctcaaaaaaaaaaaaaaaaaacccaaaaaacaaaaatgtgtaatttcatgtccttcatgggttaagatagtttttaaagaccttgccaaatatttactgagttgcctactacgatgttaagcactgttctaggtattcaggatacatcagtgaaaaaaaccgtccctgcaaccctcttaaaaaaaagtcactgtccttatggagcttactggggaggcaaataaaaaataggcaataaatagtatacaataaaagaaaatattaggtatttatcaaaatgagttgaaaacttgcacacaaatgattatagcaattttgttcataaactaccaaaaactgtccttgaagttttgatgcccttcaatatgtccatggataaactgtggtacattcatacacagtgaaatattattcagcattaagaaataagctaccaacccacaaaaagatgtggaagaaacttaatgcatgttgttaactgaaagtgcctaaaagggctccatactgtatgatcccaactatctgacattctggaaatgggtacagagtcagtaaaaacagctgggcatggtggtgcatgcctgtaatcccagctacttgagaggccaaggcggaaggactgcttgagtcccaggatatcaaaaccagctcagacaacatagcaagactccattccaaaaaaaaaaaaaagaaaagagaaaaagataacagtggttgcaaagggttggggagaagggaaaggagggatgaggcggaggagcacaagggagttttagggcattgaaactattctgcatgatactgtaatgggggagacatgccactgtgcatttttccaaatacacagaatgtacaacacaaagaatgaatgttaaactatgaaccttggttaataatgaatcaaaatcggttcatcagttgtaacaaatacaccacactaatgcatgatgttaacaggagaaactgcggggaagcggggtatatggaaattccctataatcaccgtgtattttttctgtaaacctgtaactactctaaaaaaataaagcccgttttaaaaagaacttgaaaaaataattcccccaaccaaaaatcaagtcaaataaaatcactgtcctcatgaaatttatgcgagaggcacagaagaaaacaaactaggcaataaatagtaaacaatagaagtaaaatatcccctaaggcatacaacaggcataataaaacaaaacaccatatgtacaactcccccatctccctcccactcctcatagatggtttaggctaaggttgataagtgggagaaaacttttggtttctgaaacatctcatagttaagcgtatctggatgggtgcggtggctcatgcctgtaatccccaacactttgggaggccaaggtgggtggatcacctgaggttgggagtttcagactggtctggccaatatggtgaaactctgtctttactaaaaatacaaaaattagctgggcctggtggtacatgcctgtaatcccaactactcaggaggctgaggcaggagatcacttgaacctgggaggcggaggttgtggtgagccgagatcatgccactgcactccagcctgggcgacagagcaagactctgcctcaaaaaaaaaagagtatccggttccccttcaaatatcaaattttcaaagacattaataactgaaatagaaagtgccagagaaatgctgaaagttgcttaatctagtaaatagctgaaataaataggaattttcttctttccaatcataagcaaaccagtcattaaaatctcagacacatacacacactcacacaaccccatctttgttcttgcttcaattttgttttagatacaatcgcaaccatacactctgtaataacatcactgccaaatactcaaaccacatgaaaaggcaaagtgccttttctattgtggaaataagcccatgttggggagaaatcctgggaatctcgcacaagaaacgcttatttacgtgtccagggccgcctctgccatgtttaccacaaataatactttaaacttcaaaagaaacgcacacacagtctaaggaacaatgaaggcttttatatagtccaatgaaaatgctttggaaaatgaaaagtataagaactgacagtacatggggatttttttgtctcagaatcttaatggagaatataaactataagcaggaaataggtcagatataacagcagacaaatcagtttcaagaagtcctacaaggaaaaatttgtgcaaaatgtcaggttgctaattattcattcagtaaacatttagggaatgtgccaagcagtcttctaagccctggggattcagcgacaaacaaaaacagatgcagcttctgctctcaaaagctgacatcctggctgggcgctgtggctcatgcctgtaatcccagcactttgggaggccgaggcaggcagatcacctgaggtcaggagttcaaaaccagcctggccaacgtggtgaaaccctatctctactaaaaatacaaaattagccaggtgtggtggcaggcgcctgtagtcccagctacttgagaggccgaggcaggagaatcacttcaacccgggaggtggagtttgcagcgagccaagatcgcaccactgcactccagcctgggcgaaaagagtgaaactctatctcaaaaaaaaaaaaaaaaggaagctgccatcctaatgggggaagacaatttgaacagattgaatagaaacacatgcagcaaaacagggtgatgggatgaaagcatgatgggctgctactttggtctaagcggtcaaggaaggcttctctgaggaggtgaggcctgaataacaagaaaccaggacggtgaagggcagagggaagaattctgggtgaaggggacaaggagccagaggcgctggagtggaaagagctgggcccacttggaggcacagggcggaggggctagcagcaggcgcagggagagctgctgagggggcagataggaggcaggcaggcagcacagggtttgtggggcagggcaaagtgcttaagaaaagccaaggaatggctttaagtagggtctgacatcatccgatttccatttttaaaagattactgtgctgtgaagcccagaaaggccgagagaaaacaaattacttagattacttcagcctacaggacttcacagggtgtctgtggggatttgggaagtgttagtgtcaaagtgtatctgagatctgaggccaggtgtggtggctcacgcctgtaatcccagcactctgggaggctgagataggcagataacttgaggtcaggagtttgagaccagcctggccaacatggtgaaaccccatctctactaaaaacagaaaaactagccaggcattgtggtgcgcacctgtaatcccagctactcaggaggctgaggcaggagaatcgcttgaaccagagattgcgccactgcactccagcctgggtgacagagcgagactccgttgtcttaatttaaaaaaaaaaaaaaaaaatctatctgaggcagaacaatagaacgcggcaaacgtgaagaaactttcagattctcctcccacctggcacgctgctcccctcccccatcgctctctcagctgatgacagggaaaggaagcccaggctgcaccccctcaccaaatgctgaccgccccaggcaggaaggaggctgcccttgctctcagccaaggccagctgcaccggtagtgcttgggattccaaggcctccatgcctgggactctcacctctgtccctgcccctactctcccttgtaccagagcaggacaggctctcacagcctacctttggaacagaacaggacacacatttaaaaaccctctctaaactctacaaacccagagaaagtcctactttcttgttccaagaaacctctttatcgttgttagttcttaacatcacatttctgctttggcctacttcaattgggcttctagatccaatattccaaggcaacggcttcttacaatatgtgcaaaacaaacagtcccttttctgttctcaaatgcattctaaaaggttggaatttcacaacttttcttctgctggttcctccacagtacacctaccaagcctttaccccaaggctttcttgaccctcttctctataaaatccaggccatcccatcctgtctcatgtcttcaaaaagatctacgtggcagagacattccaatctccatctcccccatgacatcttttcagaactccagactactcaacttctgtgctcttcagtccaaatggcgtctcaaaccagatggttatcagatggctacaggaggcattacggaatgtatcatcttctgaatgtgaaccacatcagatgcaatacagagaaacacacactctccaggcacagcaaaccatgccacagaacgctcgggctttacctgtgatgggaagcttgggctgcagtgtgtacagctgggcaggggtctgatgtgggttcattccatatctcaagggcatctgagatacgcctttgctgtactgtttcctgaaataatctgaaattgcttcatcatattgtgccgtatgagtgaatgcctacaaaaagcgtaagttttgtgagacacgagcagcactttccaaagtggggacaagatggttttctacgtccacagcaaacaacatctcctcactattcagtgctatgtggtggctgcacaacacgatgacaccatgaatgacagcaaagcactgaatggcccaacaaattacccctttccaattctttttcaatctttttttttttttttgagacaaagtctcactctgctgcccaggctagagtgcagtggtgtgatgtcagcttactgcaacttccacctcctgggttcaagcgattctcctgcctcagtctcccgagtagctgggattacaggcgtaccacaccacaccacactcggctaatttttgtattttcagtagagacagggtttcaccacattggccaggctggtctcaaactcctgacctcaggtgatccgcccgcctcggcctcccaaagtgctaggattacaggcgtgagccaccgcgcccagctctctttcaatctttttgacagaacacctctgtaatagctggtttctccatttttaacaaaaggatagcagctccaagcaaagctttggtaggtaatgcaatctaccaagaactgaatactgcttggtctggcaaacactgcactccatggatgtaacttacaatatcatgaaagtgcatcccaccttcaaggctaactggcgtctagtctccaaggaggtgtccttactctcggagctctgcatctccgtggacaccaccacatagtcctctggttcacacaccactgtcactcgagcgtggtttttggctgcagctctcagtaaggttactccacctttgaagaacaaaagacttattgatttttgtatgtccctatcagcttatgaaccgcttttaaataaagtcattgtgcatgtacttccattttaattcctgacttaaaaatcaggaaaataagctgggcgcagtggctcatgcctggaatcccagcactttgggaggctgaggcaggtggatcacctgaggtcaggagttcgagaccactctggccaacatggcgaaattccatctctataaaaaatacaaaaaaaattagccaggcgtggtggtgtgcacctgtaatcccagctactcaggaggctgaggcaagagaatcacttgagcccaggaggcagaggttgcagtcagccaaaactgtgccactgtactccagcctgggtgacacagcaagacttcatctcaaaaaaataaaaataaaataaataaataaataaatcaggaaaataacccagatgcatctctgcatacaaaaaaagcttagaaaatataaatgaaaaaaagaaaatataaatgaattatagtaatcccaaaacacaatccagaagtagctattttctttctatttcctcagtaataaaattcaaaatgaaatctcactaaaatttaaataatcctctcctctcagtcttctaaaatggtttttctgacttaccaatgtcaatttgctccacagcctcctcaacagttacacctggagaagccactgtctttacaaagggatagagattgcaggcaacaactctataaaacaaagacgaatctggctgtcattaaaaagtccactcatgtggtttacagctatctaggaaaaaaacaagtcagtactattaacgtaaacaaaagccatagagaactaatatataaactgtgagcctgacattttagaattctaacgagtgtgttaaaaagtcagggtgtcctgctggggtcgagcagaggtaatttagtcagcaggtgctccccggatctgaatacacagtactccctccacgtcacactgcaacgctgctgcttgcataaccttagctacccagaaccaccaagccatgaaaaatttatagaacattttaatagaagtataaagtacaagattacagataacattctcattttggaatgtgatcactattatccagaacattagaatttagattctttttccagtggattggcacaatgatgaaaaatatcagagcattctaagggtgaaacatttgttttgccttcagagaggtcataaaaatgatggtaacaggaatgccaaaagaataatttaaaatacagaagtaatctaaaattaacaatcagtgaagtacagttccactcctaggcctgtagtgcgaggagctctacgggcaggctccaaacaagcctaactgctgaaaactaattaaaaaaaacaaaaacaaaaacagaagaacagaattggaggggaaacattttgcagctcatctttattaagtctactgccctcctactaaaaccagacaaatgtcacaagaaaactagagaccagtatctcttaggtatcttcacacaaaaactctcaacaaaacactaacaaactgaatccagaaaaatgcaaaaaccacctgatcatctcaatagatgtacaaaaaggatctgacacaattcaacaggtattcatatagaaacactcagcaaattaaaaatagaagggaacttactgaacatgacaaagtgtaagaaaaacccacagctaacatcatacttgatggtgaaagactaaaatacttgtctactcaactcagaaacaagacaagaggccaggcgcagtggctcatgcctataatcccagcactttgggaggctgagccaggcagatcacctgaggtcagaagttcgagaccagcgtggccaatatggtaaaaccccatctctactaaaaatacaaaaattagccgggcaaggtggcgcacgcctgcaatcccagcactttgggaggctcaggaggatggatcaactgaggttggaagttcaagaccagcctgaccaacatggagaaaccccatctctactaaaaatacaaaattagccaggcaaggtggcacatgcctgtaaccccaggctgaagcaagataatcacttaaacccgggaggcggaggtcacagtgagccaagatcacgcaactgcactccgccttgggcaacaagagcaaaacgccatcttaaaaaaaagaaacaaaacaataatgtccattcttaccacttctactgaacactgtactagaagttctagccagagcaattagcaaagaaaagaaaataaaaggcatcaggactggaaagaaagcagtgaagttatatctatttgcagatgataccacctagtatacagaaaatcccaaattcacaaaaataaccttttagcactaataaatgagttcagcaaagccgcaggatatatgatcaatatataaaaatcaattgtattaaaatatacattagcagtgaatgagaaaaaaattaagaaaacaattctacttcacaagacaatgttaaagaaattaaagacaccccatgttcacagactggaagatttaatattcttaggatggtaacatttcccaaattgatctacagattcaacacaatacctatgaaaattccaactgcgttgttttgcagaaatggacaagctgattctaaaaatcatgtggaaatgcaagggactctgaagagtcaaagtcatcttgaataaaaaaaacgaagttggggccgggcgcagtggctcacgcctgtaatcctagcactttgggaggccgaggcgggcggatcacgaggtcaggagatcgagaccatcctggcgaacacggtgaaaccctgtctctactaaaaatacaaaaaaattagctgcgcatggtagcgggcgcctatagtcccagctactcgggaggctgaggcaggagaagggcgtgaacccgggaggcagagcttgcagtgagccaagatcgcgccactgcactccagcctgggcaacagagccagactccgtctcaaaaacaaaacaaaacaaaacaaacaaacaaacaaaaaacagaaaaatgaagttggaggactcacacttgctgatttcaaaatttgttaccagcctatagtaatagtgtagtactggcataacagcaggcacatggaccaagggtgccaagaccattcgattgggcaaagaacagtcttttcaacaaatgatgctgagacaactagacagccacatacaaaataaaataagacttctatttcatgtctgtatatgaaaattaacttaaaatgaaccaaagacttaagtataagagctaacactaaaataactcttaaaagaaatgtacacacgccgggtgcggtggctcacacttgtaatcccagcactttgggagaccgaggcaggtggatcacctgaggtcaggagttcaagaccagcctggccaacatggagaaaccccgtctctactaaaaacacaaaaaaattagctgggcgtggtggcgggcacctgtaatcacagctacttgcgaggctgaggcatgagaatcgcttgaacccaggaggcgaaggttgcagtgagccgagatcacgccactgcactccagcctgggcaacaagagcgaaactccatctcaaaaaaaaaaaaaaaaagaaagaaaatatacacaaaaatcttcatcactgaggattaggcaatggtttcttagacatgacaccattaacacaagcaacaaaagaaaaaataaattcaacatcatcaaaattaaaaacttttgtgggccgggcatggtggctcaagcctgtaatcccagcactttgggaggccaaggcgggcagataacccgaggtcaggaattcaagaccaacctggccaacatagtgaaaccctatctctattaaaaatccaaaaaaattagccaagtgtggtggcgggcacctgtaaccccagctactcatgagactgaggcagaatcgcttgaatccgggaggtggaggttgcagtgagccaagatgacgccactgtgctccagcctgggcgagagtaagactccgtctcaaaaaaaagaaaaaacttgtgatgatcaaaggaaaccatcaagaaaggctaacacaatccatagaatgagaggaaataaacttacaactcaataataaacagataaatacctcaactaaaaatggacaagaggtctcaacagacatttctccaaaggaggtaaacaactggctaggaagcacatccaacggtgttccacattatttggaaagcaaattataaccacaatgagatgccacttcagaacccctagaatgactagaatcaaaaagacagatagtaacgttgatgaggatgtggagaaactggagccccatccagcacactgctgctggaaatgtaaaatggtgcagcactctgaaatacagggtcgcagttcctcaaaagctaaacatagttaccatatgaccaaggaattccactcctaggtatataccccaatgaaacgaatgtgtatgtccacacaaaaacttatacacaaatatttatagctgcattattcataatattcaaaaagtagaaacaacccaaatgtccatcaacaaatgaactgataaacaacagggtatgaccacacaatggattatttttcagcactaaaaacgaaatgaactattgatactatgctataacatggataaaccttgaaaacatgctaagtgaaagaagccaggaacaaaaaaaccatatattgttagattctatttatatgaaaatgtccactacaggcaaatcagacacgtaacaattggtggttgccgaggctggaggacgttggggaaatggagaataaatgcgtatgggtatagggtttagggtgggggtgatgaatatgttttaatattggatgtggcaattaatatggtttggatgtgtgtcctctccaaatctcattttgcaatgtgatccccaatgttggaggtggggcctggtgggagatgattgggtcacgggggagatcccttatgaatggcttggtgccctccccataatgagtcctcactctgagttcatgtagagctggttgtttaacagagccgggcaggccgggcgtggtggctcacacctaatcccagcactttgggaggccggggtgggtgcatcatttgaggtcaggagttccaagagcagcctgaccaacatggtgaaaccccatctctactaaaaatagtaaaaatgagttgggcgtggtgacaaacgcctgtaatctcagctactcaggaggctgaggcaggagaatcacttgaacacaggaggcagaggttgcagtgagccaaggtcatgccactgaactccaacctgagcaacagagcgagaccctgtctcaaaataaaattaaattaaattaagaataaaagttaaaaagtaaaagagctgggcaactcctcctctccctctctcttgcttcttctctcatgggatatgccagctccccctctgctttccactgtgattataaacttcctgaggccctcaccagaagttgatgctggcaccacacttcttgtactgtctatagaatcacgagccaaaataaacctgttttcataaattacccagcttctagtattcctttatagcaatgcagaacagactaacacagtaatggttgcacaacttctgtgaaccactgatttgtacattttaaattggtgagttgtatggtatgtggattagatctcaataaagctgttacgtatatttaagagaaacagccagtatctttatgcaaataactcagttagttgtgcaagttgataattcctaatatgaaaacctgaaatctgaaatgctccaaaatctgaaactttttgagcgtcaacatgacacttggaggaaatgctcactgaagcatttcggatttctgagggacgctcaactggcatgttacctgcaaatattccaaaatctgaagaaatctgaaatccaagacacttctgggtcccaagcatttcagataagggatactcaacctatacatactaagatttagattgtaacttcctaaaaaacttcagtatgtattttggtatataaatctggccaaaccaaaatgcttacacacatacaaagtcacacagaaggtacgcagtaaaggtttatagtgagtaagaaaacacagggacaccaggtttggcatagtctttgtcgttggttcgtaatgtgttaaaagtttaatttcaggtttttaccttataagattgaaatcaagtctggccatgtcagcattatcttctggaatattacgagctaggattcctaatttaaaaaagttaaacaaagtcattagcttcaaaaaacaagtcagattaaggaatgtctttcaacatagtgtcttcaatcaaattaaatcccattgattaaaaaaaaaatttatttttttgagatggtctcactctatcgcccaggctggattgtagtggcgtaatcacaactcactatagccctgacctccagggctcaagtgatcctcctacctcagcctcccaagtagctgggactacaggaatgcatcaccacacccagctggtcttttcaattttttaaagagaaggggggtgttgccatcttgcccagggtgatcttaaactcctgggctcaagcggttctcctgtctcagctctccaagtgctgggactacaggcaagagccaccatgcccagcataatattcttaattaaagaatatcaaacatgtagttggggagttatcaaacaaaaaaattaggattaacagatatatctggctggccacagtggctcatgtctgtaataccagcactttgggaggctaaggtgggggatcgcttgagcccaggtgttcgagaccagcctgggcaacaaggtgagaccccgtctctataaaaaatacaaaaattagctgggtgtggtggcacatgcctatagtcccagctacttgggaggctgaggtgggagaactgcttgagcctgggaagcagacagaggctgcagcgagccatgatcgcaccactgcactccagcctgggcaagagtataatcctgtctcaaaaaaaaaaaagagagatatatttaaaaatctttttcttcagttcttgatcccagtttcttgagcaataaaatgtaaaaaaggtaaaaggaattttacaaaactgttgctagttccagaacagtctatgcgtttctgtaagtcaatggcttgtctgatgatctgtagccattcagctttattcaccaaattgtaccacaactgacaaactaggaggataccagtttcttgccgtttcggtttatatacaattcattcttaggtgaacgaacaaatgtaaagcactttgaaaaaaaaaattcctttgcaagtcagttacgctgtgatgctttcttctaagtttgaactattttaaaagtattttcatacgatgaaatctaaatatcttcagcaacttgagcaggaaacaccagactctaaattaggtatcaggtcaggactaatagcaagcagaggaaaacaatctgaattcatcaagtagtaagacaagctggattttaaaccagttctaacgtattatgaaatctggcagcaacttcataacattaactttatatgtaaatttctcttgagcctttttcttcctttattttaggacgctatcaactaaaatattcctggaaaccactgactgtttttacattaaagataccaaccacttaccagcatggactgcaggatgcaaagttttcacacgtccccccaacatttcaggaaatcccgtcaactcagagacatctctgaaaaacagataaacagaatggtactactggtgtggctgaattgtaattttcactattcaaagcatctactgttttcatttcaagagattttgagcctgaattcaacgtgaaaattctgattttttaaagtatttcactatattttatttcttaagtaaaattctatttacttccgaaaaaaagtttatttaatttatttcctaactgttgtgtatgttccgtacgattaagggcgaattccagcacactggcggccgttactagtggatccaacagtctccacgtaaacggtcaaagtccccgcggccctagacaaatattacgcgctatgagtaacacaaaattattcagatttcacttcctcttattcagttttcccgcgaaaatggccaaatcttactcggttacgcccaaatttactacaacatccgcctaaaaccgcgcgaaaattgtcacttcctgtgtacaccggcgcacaccaaaaacgtcacttttgccacatccgtcgcttacatgtgttccgccacacttgcaacatcacacttccgccacactactacgtcacccgccccgttcccacgccccgcgccacgtcacaaactccaccccctcattatcatattggcttcaatccaaaataaggtatattattgatgatgattgtttaaacatgatgagctcagc